SEQUENCE LISTING (2 sequences)

SEQ ID NO: 1 - protein, 5 aa, Plasmodium falciparum
GHHPH

SEQ ID NO: 2 - protein, 6 aa, Plasmodium falciparum
  MISC_FEATURE (6)..(6): X = Ser, His, Asn
NHKSDX

(19) United States
(12) Patent Application Publication
(10) Pub. No.: US 2003/0180396 A1
(43) Pub. Date: Sep. 25, 2003

(54) Method of effecting phosphorylation in eucaryotic cells using thiamine triphosphate

(75) Inventor: Hoang-Oanh Nghiem, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT PASTEUR, Paris (FR)

(21) Appl. No.: 10/419,802

(22) Filed: Apr. 22, 2003

Related U.S. Application Data
(62) Division of application No. 09/633,816, filed on Aug. 7, 2000.
(60) Provisional application No. 60/147,388, filed on Aug. 6, 1999.

(51) Int. Cl. A61K 35/78; A61K 31/66

(52) U.S. Cl. 424/725; 514/75

(57) ABSTRACT

A method of phosphorylation, which entails contacting procaryotic or eucaryotic cells with thiamine triphosphate (TTP) to thereby transfer a phosphate group from the TTP to a phosphate acceptor group of the cells.

This application claims the benefit of U.S. Provisional Application No. 60/147,388, filed Aug. 6, 1999.

### BACKGROUND OF THE INVENTION

1. Field of the Invention

The present invention relates to a composition suitable for treating eucaryotic cells which are under-phosphorylated, as well as a method of effecting phosphorylation in eucaryotic cells using thiamine triphosphate (TTP).

2. Background of the Invention

The neuromuscular junction (NMJ) is a sophisticated structure specialized in the transmission of neural signals from the motor nerve to the muscle cell or to the electrocyte which may be viewed as a simplified muscle cell. 43K rapsyn [rev. in (1, 2)] is a membrane-associated peripheral protein (3, 4) coextensively distributed with the nAChRs, at the inner face of the postsynaptic membrane of Torpedo electric organ (5, 6) and at rodent NMJ (7). It is necessary for nAChR clustering and formation of functional motor endplates. Mutant mice defective in 43K rapsyn gene die postnatally, display a lack of nAChR clusters and have dysfunctional postsynaptic membranes (8). In vitro removal of 43K rapsyn renders the nAChRs more mobile within the membrane plane, more susceptible to enzymatic degradation and heat denaturation (rev. in 1, 2) and more accessible to anti-nAChR antibodies (9).

Phosphorylation is important in cell signaling (rev. in 10-14). 43K rapsyn which contains several putative phosphorylation sites (15), is partially phosphorylated on serine residues in vivo and phosphorylated in vitro by endogenous protein kinase A (PKA) (16). However, this phosphorylation is not specific for 43K rapsyn and can occur with other proteins of the postsynaptic membrane (16). In view of the essential roles of phosphorylation in cell signaling (rf. in 10-14) and of 43K rapsyn in postsynaptic differentiation (rev. in 1, 2, 17), a means for effecting specific phosphorylation of this synaptic protein would be desirable.

Thiamine is essential to cell life and may play a role in the central nervous system and in synaptic transmission (18-20). The thiamine pathway includes thiamine and its mono-(TMP), di-(TDP) and triphosphate (TTP) derivatives. TTP, the non-cofactor form of thiamine, activates the maxi-Chloride channel permeability possibly via phosphorylation (21). Low concentrations of TTP are found in most cells (22) except in neuronal (23), and excitable (24-26) cells. However, at present, it is unknown whether TTP could effect a specific phosphorylation of 43K rapsyn, let alone phosphorylation of other proteins in cells.

### SUMMARY OF THE INVENTION

According, it has now been surprisingly discovered that [γ⁻³²P]-labeled thiamine triphosphate ([γ⁻³²P]-TTP) functions as a donor of phosphate for proteins present in the acetylcholine receptor (nAChR) enriched postsynaptic membranes purified from Torpedo electric organs, for example. Electrocytes which can be considered as simplified muscular cells have been used as a model system for the neuromuscular junction (NMJ). When incubated with such purified AChR-enriched postsynaptic membranes, [γ⁻³²P]-ATP (adenosine triphosphate) used as a phosphodonor leads to phosphorylation of many proteins. On the contrary, [γ⁻³²P]-TTP leads to a specific phosphorylation of 43K rapsyn, a synaptic cytoskeletal protein present in the postsynaptic membrane and essential for AChR clustering and aggregation, and for the formation of functional end plates at the NMJ. Thus, the present invention represents the first utilization of TTP as a phosphodonor and affords a strong specificity of TTP-dependent phosphorylation for target proteins.

Preferably, this phosphorylation occurs predominantly on histidine residues and is catalyzed by new endogenous protein kinase(s). Phosphorylations on histidine residues have been mostly observed in procaryotes and simple eucaryotes. They usually participate in the regulation of cellular functions through histidine protein kinases. However, they also occur in higher eucaryotic cells and the existence of the enzyme nucleoside diphosphate kinase (NDPK) which plays a key role in growth and metastasis control shows the putative importance of phosphorylations on histidine. Few cases of phosphorylations on histidine residues have been reported in higher eucaryotes, probably as a result of a massive use of ATP as phosphodonor and of the availability of analytical methods more suitable for O-phosphorylations than for N-phosphorylations (phosphohistidines are N-phosphorylated residues). This clearly indicates a need to elucidate techniques more adapted to N-phosphorylations amide bonds) and of investigations on such novel phosphorylation reactions which would elucidate a phosphorylation pathway important for synaptic and/or eucaryotic proteins.

In accordance with the present invention, the use of TTP as a phosphodonor is explicitly extended to proteins of other cellular systems as a means for analzying the effect of such TTP-dependent phosphorylations. In particular, extension of the use of TTP to the neuronal, immune and endocrine systems is explicitly contemplated. In using TTP as a phosphodonor for proteins in rat or mouse crude brain membrane preparations, the present inventor has observed that some proteins are phosphorylated with endogenous kinases present in the preparations. Analysis of TTP-dependent phosphorylations of brain membrane and of cytoskeletal proteins has been investigated. The present inventor has also extended this analysis to muscle and to spinal cord proteins from birds and mammals, in particular, from rats, mice, monkeys and humans. Moreover, the present inventor has also extended this analysis to the immune and endocrine systems. Hence, the present invention has broad applicability.

This use of TTP as phosphodonor, in accordance with the present invention, opens up a new area in the field of phosphorylation which will provide for a better understanding of the role of TTP-dependent phosphorylations in the physiological cellular processes. If, as now appears to be the case, these phosphorylations are crucial for cell functions, their evaluation will lead to a better understanding of diseases derive from a dysfunction of molecules involved in TTP-dependent phosphorylations and are most important for therapeutics.

Purification, sequencing and cloning of the histidine kinases involved in the TTP-dependent phosphorylation of proteins using known methodologies also allows their classification in new proteins using known methodologies kinase family or as members of known kinase families. This will also provide insights in the role of the new kinases in the regulation of cellular processes especially in neuronal and muscular cells.

The present invention also enables further therapeutic analysis of diseases linked to a deficit in TTP-dependent phosphorylations or to TTP-dependent hyperphosphorylations of proteins essential to the regulation of critical functions in the cell.

In addition, while it is already known that thiamine is involved in cognitive impairment of the nervous system (U.S. Pat. No. 5,885,608 and U.S. Pat. No. 5,843,469), the results obtained were not related to the use of thiamine triphosphate.

However, the present invention demonstrates, for example, that in the presence of radiolabeled TTP, Torpedo 43K rapsyn is the predominant protein phosphorylated by endogenous kinase(s) present in nAChR-rich postsynaptic membrane preparations. Phosphorylation occurs mostly at histidine(s) and at some serine(s). Both TTP- and ATP-dependent phosphorylations of 43K rapsyn are inhibited by TTP and ATP. TTP-dependent kinase(s) might thus share some phosphorylation site(s) with PKA. The likely regulation of 43K rapsyn phosphorylation by endogenous Zn²⁺ and the modulation of 43K rapsyn functions via its phosphorylation state is discussed below. The extension of phosphorylation to rodent brain membranes suggests a more general use of TTP as phosphate donor for synaptic proteins as well as a novel phosphorylation pathway. The present invention is predicated upon this broad and general discovery.

Thus, the present invention provides, in part, a composition suitable for treating eucaryotic cells which are under-phosphorylated, containing an effective amount of thiamine triphosphate to increase the phosphorylation level of the cells. This composition may also contain other components customarily added to cell-treating compositions, such as buffers, electrolytes, cellular nutrients, anti-oxidants, etc., and may be in any form suitable for in viro administration, such as in tramuscular or intravenous, for example.

The present invention also relates to use of TTP in a method of preparing a composition containing TTP for the treatment of a mammal, preferably a human patient, having or exhibiting a pathology, ie. A disease or condition or symptoms, associated with an under-phosphorylation of a post-synaptic protein or having a deficit in the formation of functional motor endplates. This composition contains TTP in combination with a pharmaceutically acceptable carrier or diluent. Any carrier or diluent may be used which is conventionally used for vitamins, particularly B vitamins. However, the composition of the present invention does not include vitamins, particularly B vitamins, however it may contain, if desired, one or more other phosphorylating agents conventionally known to those skilled in the art.

The present invention also relates to the use of TTP in the preparation of a composition for treating cell membranes or cytoskeleton of cells which are deficient in phosphorylated histidine residues. Preferably, the histidine residues are on the rapsyn protein.

The present invention also relates to a method of treating a patient who has a pathology associated with underphosphorylation of a post-synaptic protein or having a deficit in the formation of functional motor endplates, which entails administering an effective amount of thiamine triphosphate to the patient. In a preferred embodiment, the thiamine triphosphate is administered in the form of a pharmaceutically acceptable composition containing the thiamine triphosphate and a pharmaceutically acceptable carrier or diluent.

Another aspect of the present invention entails a method of treating cell membranes which are deficient in phosphorylated histidine residues, such as rapsyn, in vivo, which entails contacting the membranes with an amount of thiamine triphosphate effective to increase the amount of phosphorylated rapsyn.

This method may be used in the treatment of cells to improve a neuronal or muscular function.

The present invention also includes a method of phosphorylating rapsyn, comprising contacting rapsyn with thiamine triphosphate to phosphorylate the rapsyn. This method may be performed in vitro or in vivo by administering an effective amount of thiamine triphosphate to a subject, e.g., a patient. The subject may be a human or an animal. Human subjects or patients are especially preferred. In fact, for all of the methods of the present invention, the subject or patient may be a human or an animal, with a human subject or patient especially preferred.

The present invention also includes a kit for detecting the specific phosphorylation of histidine residues in a protein, comprising:

(a) radioactively labeled thiamine triphosphate,

(b) non-radioactively labeled thiamine triphosphate,

(c) reagent(s) for transfer of the thiamine triphosphate,

(d) a purified protein containing TTP dependent phosphorylatable histidine residues,

(e) a protein containing non TTP-dependent phosphorylatable histidine residues, and

(f) optionally, antiphosphoamino acid antibodies.

In the kit, (d) serves as a positive control and (e) serves as a negative control.

Another aspect of the present invention is a method of quantifying the level of phosphorylation of the membranes of eucaryotic cells, comprising:

(a) purifying the membranes from a eucaryotic cell sample obtained from a patient,

(b) incubating the membranes with thiamine triphosphate,

(c) comparing the incorporation of exogenous phosphate with a control, and

(d) determining the presence or absence of phosphorylated histidine residues in a protein in the sample.

Also included in the present invention is a method of phosphorylation, entailing contacting procaryotic or eucaryotic cells with thiamine triphosphate to transfer a phosphate group from the thiamine triphosphate to a phosphate acceptor group of the cells. In a preferred embodiment, the phosphate acceptor group of the cells is a histidine residue of a cellular protein.

The present invention also includes a kit for detecting the specific phosphorylation of histidine residues in a protein, containing:

(a) radioactively labeled thiamine triphosphate,

(b) non-radioactively labeled thiamine triphosphate,

(c) reagent(s) for transfer of the thiamine triphosphate,

(d) a purified protein containing TTP dependent phosphorylatable histidine residues,

(e) a protein containing non TTP-dependent phosphorylatable histidine residues, and

(f) optionally, antiphosphoamino acid antibodies.

In the kit, (d) serves as a positive control and (e) serves as a negative control.

Another aspect of the present invention is a method of quantifying the level of phosphorylation of the membranes of eucaryotic cells, containing:

(a) purifying the membranes from a eucaryotic cell sample obtained from a patient,

(b) incubating the membranes with thiamine triphosphate,

(c) comparing the incorporation of exogenous phosphate with a control, and

(d) determining the presence or absence of phosphorylated histidine residues in a protein in the sample.

Also included in the present invention is a method of phosphorylation, containing contacting procaryotic or eucaryotic cells with thiamine triphosphate to transfer a phosphate group from the thiamine triphosphate to a phosphate acceptor group of the cells. In a preferred embodiment, the phosphate acceptor group of the cells is a histidine residue of a cellular protein.

The present invention also includes a process for the purification of a new type of kinases and said purified protein extract carrying a TTP dependent kinase activity.

The process of purification entails the following steps:

extraction from eucaryotic tissues by homogenization in buffers containing a cocktail of inhibitors of proteases (antipain^{R}, aprotinin^{R}, leupeptin^{R}, pepstatin A^{R}, EDTA, EGTA for example).

centrifugation at low speed (Beckman JA10 rotor; 5K rpm, 10 minutes) at 4° C. to collect the cellular extract in the supernatant.

Centrifugation (Beckman JA14 rotor; 12.5K rpm, 50 minutes, 4° C.) of the cellular extract to collect the pellet as the membrane fraction.

The membrane pellet is resuspended in the homogenization buffer and adjusted to 35% sucrose (w/w) by addition of sucrose.

Ultracentrifugation at high speed on a discontinuous sucrose density gradient (35% and 43% sucrose) of the membrane suspension (Beckman 45Ti rotor; 40K rpm for 3 hours, 4° C.).

The purified membranes were recovered at the 35%/43% sucrose interface and collected by centrifugation at 40K rpm.

Optionally, the membrane fraction is further purified on a continuous (35% to 43%) sucrose density gradient (Beckman SW27 rotor; 18K rpm for 12 hours, 4° C.).

The collected band contains the new TTP-dependent protein kinase activity.

The purified protein extract has a KD comprised between 5 μm TTP and 25 μm TTP.

The kinase activity of the purified protein extract, is favored by pH around 7.5.

The invention will now be described in more detail, and in reference to the Figures described below.

### BRIEF DESCRIPTION OF THE DRAWINGS

FIG. 1: Phosphorylation of a 43 kDa protein with TTP as the phosphate donor by endogenous kinase(s) present in the nAChR-rich postsynaptic membrane. 1A: SDS-PAGE-autoradiogram of electrocyte postsynaptic membranes phosphorylated with 8 μM[γ-³²P]-TTP in the presence of various effectors reveals one major radioactive band at ˜43 kDa (arrow). Phosphorylation is inhibited by cold TTP in a dose-dependent manner [24 μM (lane 8/control lanes 4,9) and 240 μM (lane 7/control lanes 4,9 and lane 5/control lanes 3,6)]. Molecular mass markers: far right. 1B: A sister gel of FIG. 1A was blotted and split into two parts. Lanes 1-3 were incubated with ¹²⁵I-Bgtx, lanes 4-9 with buffer. Both parts were realigned and autoradiographed. In lane 2, where phosphorylation had been prevented, the radioactive band observed with ¹²⁵I-Bgtx is α-nAChR (arrow head). In lanes 4-9, the radioactive band is the ³²P-labeled protein at 43 kDa (arrow). In lane 3, where the ³²P-membrane has been further incubated with ¹²⁵I-Bgtx, two radioactive bands are observed demonstrating that the TTP-dependent phosphorylated 43 kDa protein is not α-nAChR. 1C: Coomassie blue of FIG. 1A autoradiogram.

FIG. 2: The TTP-dependent ³²P-phosphorylated 43 kDa protein is 43K rapsyn. 2A: Postsynaptic membranes phosphorylated in the presence of ³²P-TTP were solubilized and immunoprecipitated with three specific anti-43K rapsyn anti-peptide antibodies (lanes 1-3). No radioactivity was precipitated with preimmuneserum (lane 4). 2B: Immunoprecipitation with increasing volumes of anti-43K rapsyn shows that the immunoprecipitated radioactivity is proportional to the amount of antibodies used (lanes 3-5). The specificity of the immunoprecipitation is demonstrated with preimmuneserum and preabsorbed anti-43K rapsyn antibodies (lanes 1,2). 2C: The ³²P-radioactivity remaining in supernatants of immunoprecipitation decreases proportionally to the amounts of antibody added, indicating that most if not all radioactive phosphate is on 43K rapsyn.

FIG. 3: Characteristics of the endogenous TTP-dependent kinase(s) which catalyze(s) phosphorylation(s) of 43K rapsyn. 3A : Inhibition of TTP-dependent phosphorylation of 43K rapsyn by TTP, ATP and GTP triphosphates. 3B: The TTP-kinase activity is optimum at light alkaline pH. 3C: 43K-rapsyn phosphorylation is dose dependent and saturable (K_{D}˜5-10 μM TTP). 3D: Kinetics of TTP-dependent phosphorylation of 43K rapsyn.

FIG. 4: The TTP-dependent kinase which specifically phosphorylates 43K rapsyn is different from ATP-dependent kinases. Autoradiogram of postsynaptic membranes phosphorylated with ³²P-TTP or ³²P-ATP shows that with ³²P-TTP only 43K rapsyn (arrow) is phosphorylated (lanes 1-2) while with ³²P-ATP (lanes 3-6) many proteins including nAChR subunits and 43K rapsyn are phosphorylated. This suggests the involvement of different kinases depending on the nature of the phosphodonor.

FIG. 5: Analysis with anti-phosphoamino acid antibodies. Similar amounts of control (Mb) and ³²P-TTP-dependent labeled (³²P-Mb) postsynaptic membranes were electroblotted. 43K rapsyn (arrow) and α-nAChR (arrow head) were marked for identification (dots •). Blots were probed with antibodies at dilutions proposed by the manufacturer: anti-phosphotyrosine (PY 1: 2000), anti-phosphothreonine (PT 1: 50) and anti-phosphoserine (PS 1: 500). 5A : Anti-PY stained various proteins but not 43K rapsyn in both membranes (lanes 1,5). Anti-PT faintly stained some protein bands but not 43K rapsyn (lanes 2,6). Anti-PS slightly stained 43K rapsyn of both membranes (lanes 3,7): note the higher signal on ³²P-43K rapsyn (lane 7). This suggests the presence of in situ phosphorylated serine on 43K rapsyn and of ³²P-phosphorylated serine brought about by TTP-dependent kinase(s). Normal serum in lanes 4,8. 5B: Ponceau red staining of control (lane 9) and ³²P-labeled-membranes (³²P-Mb, lane 10) shows slightly less proteins in ³²P-Mb. 5C: Immunostaining of ³²P-labeled membrane with anti-PS antibodies has been repeated and confirmed ³²P-43K rapsyn staining by anti-PS antibodies (lanes 11,12). Control in lane 13.

FIG. 6: Phosphoamino acid analysis of phosphorylated 43K rapsyn. 43K rapsyn phosphorylated by ³²P-ATP or ³²P-TTP were separated by SDS-PAGE, blotted onto PVDF, hydrolyzed in 5.7N HCl (1 h, 105° C.) and analyzed for phosphoamino acids by 1D-high voltage electrophoresis on thin layer cellulose in pH 3.5 solvent. Non radioactive P-Ser, P-Thr and P-Tyr (lane 2) were run as standards. The ATP-dependent ³²P-43K rapsyn hydrolysate (lane 1) shows several main radioactive spots stained by ninhydrin (dotted lines) at phosphopeptide regions and at P-Ser level. This is consistent with serine phosphorylation reported in (16). The TTP-dependent ³²P-43K rapsyn hydrolysate leads to a similar ninhydrin-stained pattern (lane 3, dotted lines) but a quite different radioactivity pattern with little radioactivity at phosphopeptide regions, a very faint radioactivity at P-Ser level, and most of the radioactivity at the inorganic phosphate (Pi) region (lane 3). These results show that ATP and TTP drive different phosphorylations on 43K rapsyn.

FIG. 7: TLC analysis of TTP-³²P-43K rapsyn. Alkaline hydrolysates (3N KOH, 1 h, 105° C.) of TTP-³²P-43K rapsyn and ATP-³²P-NDPK, and trypsin/pronase digest of TTP-³²P-43K rapsyn were resolved by TLC in solvent A, stained with ninhydrin (dotted lines) and autoradiographed. External standards were P-Ser (lanes 1), P-His (lanes 5). 7A : Trypsin/pronase digest (lane 2), alkaline hydrolysates of TTP-³²P rapsyn (lane 3) and of ³²P-NDPK (lane 4) all show radioactivity at P-His level. 7B: P-His (dotted circle) added to alkaline hydrolysates of TTP-³²P-43K rapsyn (lanes 2,3) or of NDPK (lane 4) comigrates with the radioactive spot. This strongly suggests histidine phosphorylation driven by TTP in 43K rapsyn.

FIG. 8. TTP is a phosphodonor for brain membrane proteins. Rodent crude brain membrane extracts incubated with ³²P-ATP (FIG. 8A) and ³²P-TTP (FIG. 8B) were analyzed by SDS-PAGE and autoradiography (molecular mass markers: far left). Torpedo postsynaptic membranes were used as controls. 8A: Torpedo ATP-³²P-membranes (lane 1). ATP phosphorylates many proteins in brain membrane extracts (lane 2) and phosphorylation is inhibited by cold ATP (lane 3). 8B: Brain membranes incubated with ³²P-TTP offers a much simpler radioactive pattern with two major ³²P-bands around 46 kDa (lane 2). Phosphorylations are partly inhibited by cold TTP (lane 3). Lane 1: control Torpedo TTP-³²P-membranes were incubated with low concentrations of ³²P-TTP to match the weak brain membrane signals.

FIG. 9A shows an autoradiogram obtained after TTP-dependent phosphorylation of membranes from different tissues. The regions under 30 kDa have not beeb examined. Molecular markers were indicated in the far left lane.

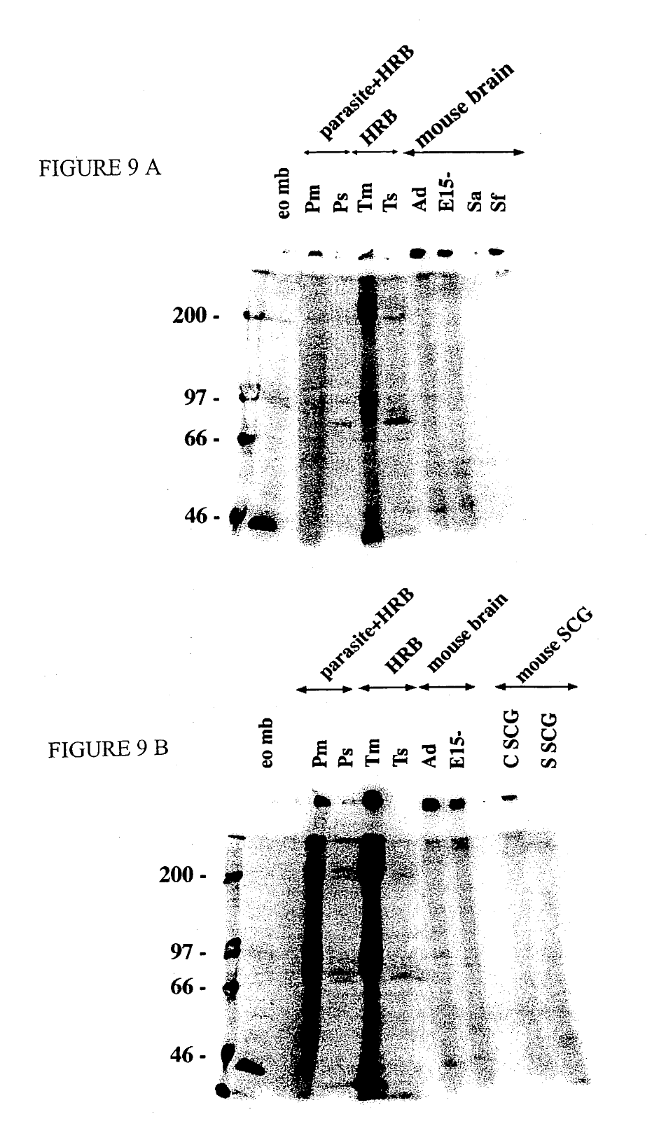

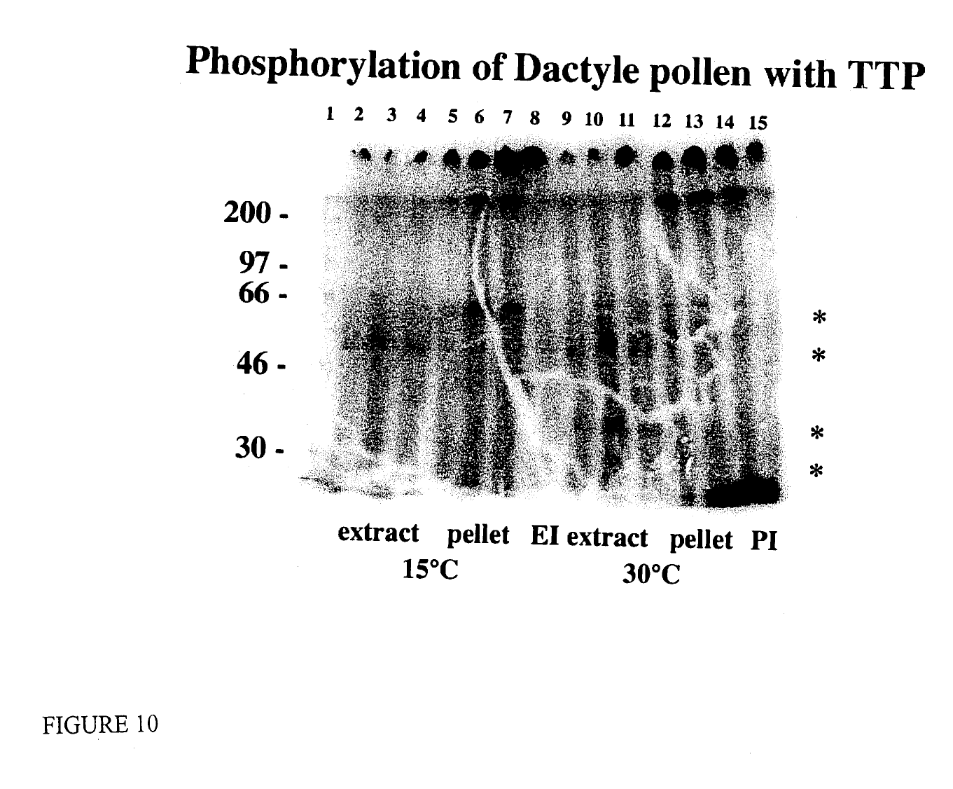

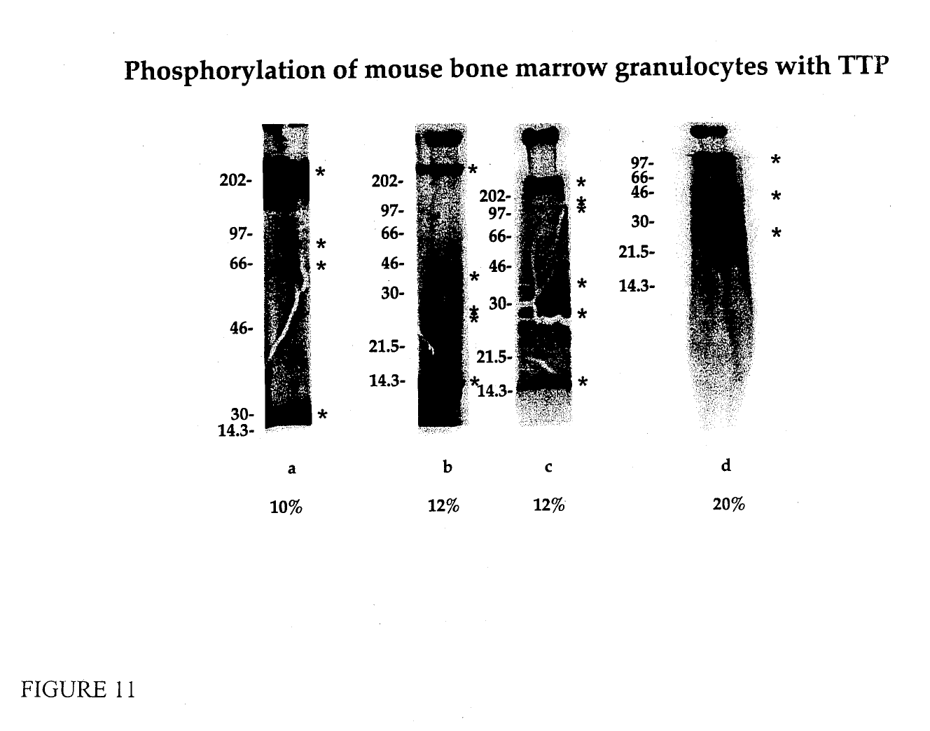

Phosphorylation of proteins from human red blood cell (HRB) membrane (Tm) occurs mainly at bands around 30-40 kDa, 70 kDa and 200 kDa). The HRD lysate (Ts) shows at least three phosphorylated bands, one around 66 kDa, and two highly phosphorylated bands in the 70 and 200 kDa regions). Comparison between phosphorylations in fractions Pm (parasite+red blood cell membrane) and Tm (human blood cell membrane) shows that the phosphorylated protein bands which are detected only in the Pm fraction (lane Pm, see for instance bands around 50, 55-60, 100, between 100 and 201 kDa, FIGS. 9a and 9b) should derive from the P. falciparum parasite. The phosphorylation patterns in lysates Ps and Ts are two weak to allow a clear cut answer. (In FIG. 9b the amount of proteins in Pm and Tm has been doubled compared to the same fractions in FIG. 9a).

Adult mouse brain membrane (A) shows phosphorylated proteins mostly at the 46-50 and 100 kDa regions. Phosphorylations can also be observed with 15 day-embryonic mouse brain mmbranes (E15, FIGS. 9a and 9b). The two phosphorylation patterns between Ad and E15 are not identical and might be due to age differences of the brain fractions.

Mouse stem cell neurosphers (Sa and Sf) showed phosphorylation at the 50-60 kDa regions (FIG. 9a).

Mouse superior cervical ganglion membranes (CSCG) showed phosphorylation between the 40-66 kDa regions and supernates (SSCG) were also phosphorylated with TTP (phosphorylated bands between the 30 to 97 kDa).

In FIG. 9b Dactyle pollen membrane proteins are phosphorylated mainly at the 30 and 55-60 kDa regions (C pollen). With the pollen lysate fraction (5 pollen), a main phosphorylated band was observed at the 55-60 kDa region (see also FIG. 10).

Control phosphorylations were performed with electrocyte membranes (co mb).

FIG. 10 shows an autoradiogram obtained after phosphorylation with 32P-TTP of Dactyle pollen proteins. Proteins in the 30 to 66 kDa regions (*) are phosphorylated at 15° C. and at 30° C. with 32P-TTP by endogenous kinases both in the water-extract (lanes 2 to 4; 9 to 11) and the pellet fractions (lanes 5 to 7; 12 to 14). Specificity of the TTP-dependent phosphorylation of the water-extract (lane E1) and of the pellet (lane PI fractions are demonstrated by a decrease of the phosphorylation upon preincubation with cold TTP.

FIG. 11 shows TTP-dependent phosphorylation of mouse bone marrow granulocytes. In FIG. 11a, right lane (SDS-PAGE 10% acrylamide), most of the phosphorylation of the pellet fraction (C2K, obtained by centrifugation at 200×g of homogenates of granulocyte cytoplasts) resided in band at very high molecular weight (*). Phosaphorylated bands were also detected around the 66- and 97 kDa region (*). A high degree of phosphorylation was also detected at the 14.5-30 kDa region (*). This band has been extracted and further characterized by autoradiography in 12% (FIG. 11b and FIG. 11c) and 20% acrylamide (FIG. 11d SDS-PAGE gels. FIGS. 11b and 11c showed major phosphorylation bands around 25 kDa(*). FIG. 11d showed phosphorylation at the 25 kDa region (*) but also at the 30-46 kDa region (*).

### DETAILED DESCRIPTION OF THE PREFERRED EMBODIMENTS

Materials and Methods

Postsynaptic Membranes

nAChR-rich postsynaptic membranes (nAChR-membranes) were prepared from electric organs excised from freshly killed Torpedo marmorata (T.m.) (Biologie Marine, Arcachon) (3, 16).

Phosphate Donors, Phosphorylation and Quantification

[γ-³²P]-ATP (³²P-ATP) was from ICN. [γ-³²P]-TTP (³²P-TTP) was synthesized (27). nAChR-membranes were phosphorylated with (7-8000 Ci/mol) ³²P-TTP or ³²P-ATP in 50 mM Tris-HCl pH 7.5, 5-15 mM MgCl₂, 0.08% CHAPS, inhibitors of proteases at 4°-20° C. for 60-90 minutes. Phosphorylation was stopped with SDS-sample buffer. ³²P-phosphorylated membranes were subjected to SDS-PAGE designed to separate actin, 43K rapsyn and α-nAChR, and autoradiographed (Kodak Biomax) and/or ³²P-quantified (Molecular Dynamics phosphorimager). Coomassie blue staining was performed when necessary. Where specified, nAChR-membranes were treated with 5-20 mM diethylpyrocarbonate (DEPC.) (28) in 50 mM Na phosphate buffer pH 6.0 and 7.4 (20 min, 16° C.), prior to incubation with ³²P-TTP. Common kinase effectors [cAMP; Adenosine 3′-5′-cyclic monophosphate, 8-(4-Chlorophenylthio)-sodium salt (8-CPT-cAMP) ; anisomycin; cGMP; calmidazolium; calphostin; cdc2 peptide; genistein; bisindolylmaleimide I (GFX); H7; H89; KN62; KT5720, ML7; protein kinase A inhibitor (PKI); staurosporine; tumor necrosis factor-alpha (TNF-α); phorbol-12-myristate-13-acetate (TPA)] were tested for their effects on TTP-dependent phosphorylation of 43K rapsyn.

Chemical Stability and Nature of the Phosphate Links

For acid treatment, SDS-PAGE gels containing ³²P-ATP- or ³²P-TTP-treated membranes were cut and incubated with Tris buffer or 16% TCA at 90° C. (29, 30), washed, and analyzed. Equivalent amounts of 43K rapsyn were ensured by Coomassie blue staining. For base treatment, ³²P-labeled membranes were resolved by SDS-PAGE, electroblotted on polyvinylidene difluoride membrane (PVDF). Blots were dried at 55° C. to minimize protein loss, wet in methanol, washed with H₂O, cut and incubated in water or 1N KOH at 46° C. and analyzed.

Phosphoamino Acid Analysis on PVDF-Electrotransferred (31)³²P-43K Rapsyn

For the determination of acid-stable phosphoamino acids, 43K rapsyn was hydrolyzed with 40 μl 5.7N HCl (1 h, 105° C.). The supernatant was evaporated and 10 μl H₂O was added. Hydrolysates were analyzed by either 1D-(pH 3.5) or 2D-high voltage electrophoresis on a thin layer cellulose plate (first electrophoresis, pH 1.9; second electrophoresis, pH 3.5) (32). For a base-stable analysis, 43K rapsyn was hydrolyzed in 3N KOH (3h, 105° C.), neutralized with 10% HClO₄ to pH 7.5 (33). Supernatants were analyzed by thin layer chromatography (TLC) on silica gel 60A° plates (ICN) in solvent A (t-butanol:methyl ethyl ketone:acetone:methanol:water:concentrated NH₄OH, 10:20:20:5:40:5, v:v) which separates phosphohistidine (P-His) from phosphoserine (P-Ser) and phospholysine (P-Lys) (33). Phosphohistidine and phospholysine were synthesized from polyhistidine and polylysine respectively (34). Enzymatic hydrolysis was conducted with 2 μg TPCK-trypsin (Promega) in 40 μl of trypsin buffer (10 mM NaHCO₃, 135 mM NaCl, 0.1% SDS, 1 mM CaCl₂, pH 8.5) (90 min, 37° C.). 2 μg TPCK-trypsin was added (2 h, 37° C.) followed by 400 μg pronase (Boehringer-Mannheim) (18 h, 37° C.). Supernatants were analyzed by TLC in solvent A. Phosphoamino acids and phosphopeptides were vizualized with ninhydrin.

Phosphopeptides

Phosphopeptides were generated by tryptic digestion on PVDF-transferred ³²P-43K rapsyn with TPCK-trypsin (o.n.; 37° C. in trypsin buffer). Hydrolysates were resolved in 15% SDS-PAGE and autoradiographed for ³²P-peptide identification.

Labeling With Antibodies or α-Bungarotoxin (Bgtx)

³²P-membranes were resolved by SDS-PAGE, electroblotted (35), treated according to (36) and probed with specific anti-43K rapsyn (37), specific anti-phosphoamino acid antibodies (Sigma), or ¹²⁵I-Bgtx (Amersham) and analyzed.

Immunoprecipitation

³²P-membranes were diluted into 1 ml 50 mM Tris-HCl pH 8.8/0.1% SDS/1% NP40/0.5% deoxycholate/protease inhibitors/0.15M NaCl, precleared with 50 μl protein A-agarose beads (Santa Cruz) and immunoprecipitated with anti-43K rapsyn anti-peptide antibodies which specifically recognize 43K rapsyn (37). 30 μl of protein A beads were added (o.n., 4° C.). Beads were centrifuged, washed and analyzed.

Results

The TTP-Dependent Phosphorylated Protein is 43K Rapsyn

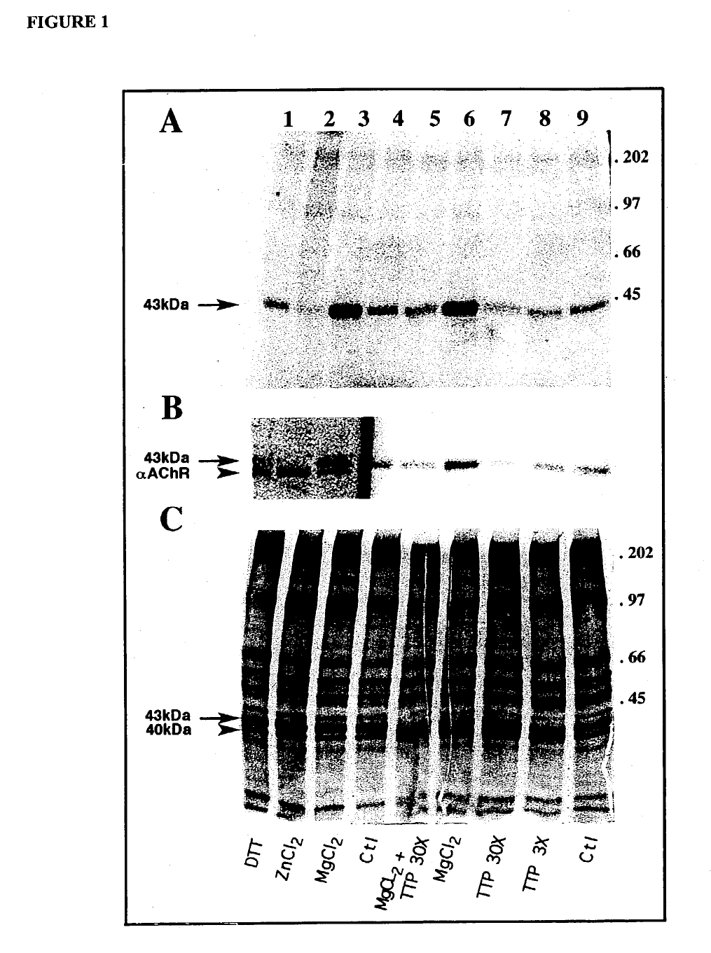

Protein(s) phosphorylated upon incubation of nAChR-membranes with [γ-³²P]-TTP migrated at ˜43 kDa (FIG. 1, arrow). The phosphorylation occurs without externally added kinases, is enhanced by Mg²⁺ (5 mM, FIG. 1A, lanes 3,6), partially inhibited by DTT (FIG. 1A, lane 1), inhibited by Zn²⁺ (FIG. 1A, lane 2), and TTP in a dose-dependent manner [FIG. 1A: 24 μM (lane 8 versus 4,9); 240 μM (lane 5 versus 3,6; lane 7 versus 4,9)].

Upon further incubation of the blotted ³²P-labeled membrane with ¹²⁵I-Bgtx, a toxin specific for α-nAChR, two radioactive bands were observed (FIG. 1B, lanes 1,3). In lane 2, where phosphorylation had been prevented, only one radioactive band corresponding to the ¹²⁵I-Bgtx-labeled band (arrow head) and distinct from the ³²P-labeled 43 kDa band (arrow) was observed. This demonstrates that α-nAChR is not phosphorylated by ³²P-TTP.

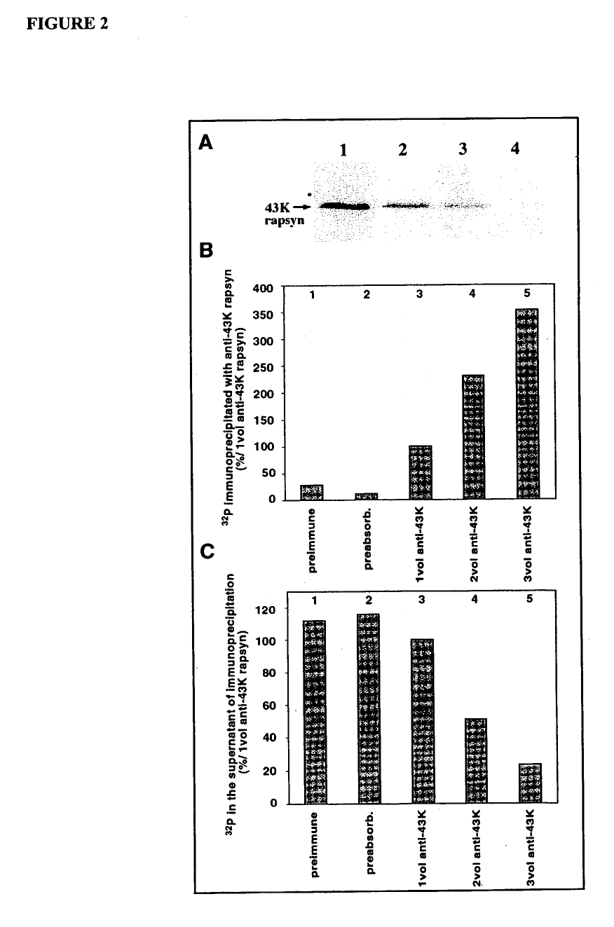

The ³²P-labeled band was recognized by anti-43K rapsyn antibodies (immunoblot, data not shown). To ascertain that the ³²P-phosphorylated protein is 43K rapsyn, immunoprecipitation of ³²P-labeled membranes was conducted with three specific anti-43K rapsyn anti-peptide antibodies (37). FIG. 2A shows that the ³²P-labeled protein was specifically immunoprecipitated by anti-43K rapsyn antibodies. One anti-43K rapsyn antibody (FIG. 2A, lane 1) used in a semi-quantitative analysis (FIG. 2B) showed that the radioactivity immunoprecipitated is directly correlated to the amount of anti-43K rapsyn used (FIG. 2B, lanes 3-5). Supernatants of immunoprecipitation showed the opposite situation (FIG. 2C). The specificity of the immunoprecipitation was verified with preimmuneserum and preabsorbed antibodies (FIG. 2B, lanes 1,2). This demonstrates that 43K rapsyn is the TTP-dependent phosphorylated protein.

The TTP-Dependent-Phosphorylation is Driven by Endogenous Kinase(s) Present in the nAChR-Rich Membranes

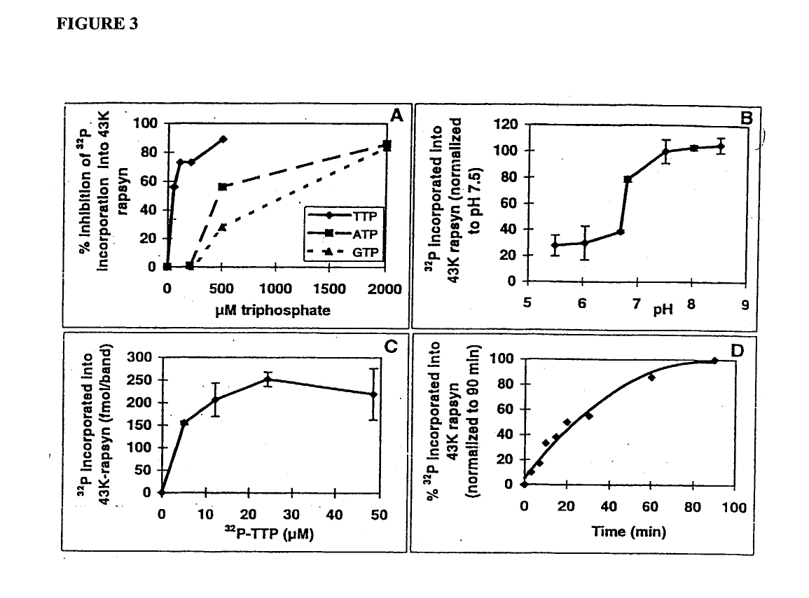

The phosphorylation of 43K rapsyn which occurs at 4-22° C. without externally added kinases, Mg²⁺-(5 mM, FIG. 1A, lanes 3,6), pH-, and time-dependent (FIG. 3). It requires TTP, is dose dependent and saturable (K_{D}˜5-10 μM TTP, FIG. 3C; however with one membrane preparation K_{D}˜25 μM TTP) and presents characteristics of an enzymatic reaction. Thus TTP-dependent phosphorylation of 43K rapsyn is driven by endogenous kinase(s) copurified with the postsynaptic membrane. The IC₅₀ for TTP, ATP, GTP are around 40 μM, 500 μM, and 1000 μM respectively (FIG. 3A). CTP inhibits poorly. The TTP-dependent kinase or kinases (TTP-kinase, TTP-43K-kinase) activity is favored by light alkaline pH (FIG. 3B) and partially inhibited by DTT (30-40% inhibition/10 mM; FIG. 1A, lane 1).

TTP-43K-Kinase is not PKA

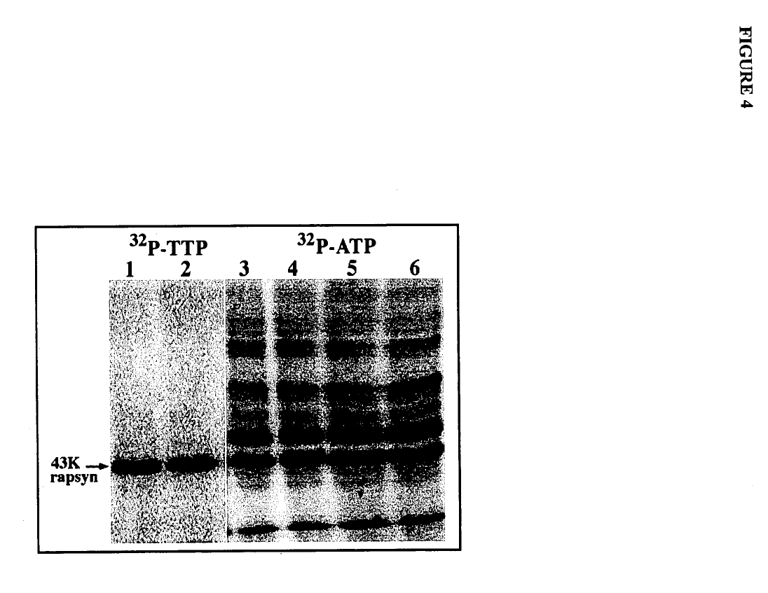

While 43K rapsyn is, within the detection sensitivity, the only protein phosphorylated in the presence of ³²P-TTP (FIGS. 1; and 4, lanes 1,2), additional proteins, including nAChR subunits, are phosphorylated with ³²P-ATP (FIG. 4, lanes 3-6) in agreement with our former results (16). 43K rapsyn phosphorylation is saturated with ˜25-50 μM ³²P-TTP (FIG. 3C) while it is not yet saturated with 200 μM ³²P-ATP. ³²P-ATP- and ³²P-TTP-dependent 43K rapsyn phosphorylation are specifically inhibited by both TTP and ATP suggesting the presence of common phosphorylation sites between PKA and TTP-kinase. However analysis conducted with PKA effectors showed that they are different. PKI inhibited PKA (60±13% inhibition) but not TTP-kinase (6±1% inhibition). Exogenous PKA catalytic subunit increased the ATP-dependent phosphorylation (603±14% ³²P versus 100±23% in control) (16, this study) while inhibiting that driven by TTP (41±6% versus 100±2% in control).

TTP-43K-Kinase, a Novel Kinase

Putative phosphorylation sites (15) for PKA [Ser-406 (38)] and tyrosine kinase [Tyr-98, Tyr-189, Tyr-325 (39)] are present on Torpedo 43K rapsyn. Searches on Prosite (40), and PhosphoBase (41) showed putative sites for CaMII, CKI, CKII, PKA, PKC and PKG protein kinases. Out of eighteen common kinase effectors, only staurosporine caused a slight inhibition (33±3% inhibition/200 nM). Numerous activators or inhibitors of PKA, PKC (TPA, calphostin, GFX), MAP kinase, Protein kinase G, CaM kinase II, JNK2 kinase, cdc2 kinase, MLCK, SAP kinase, and TyrPK (data not shown) did not drastically alter the activity of TTP-kinase which is likely of a novel type.

This TTP dependent phosphorylation is catalyzed by at least a new endogenous kinase. This kinase is copurified as disclosed supra and is characterized by the determination of the KD (apparent dissociation constant shown on FIG. 3C) and by the IC50 (product concentration giving 50% of inhibition of the enzymatic activity of saiol kinase in presence of TTP or ATP or GTP as shown on FIG. 3A). The kinase is also pH dependent. The optimal pH is around 7.5. A purified extract containing the kinase responsible for the TTP dependent phosphorylation of histidine residues on rapsyn, is preincubated with increasing concentration of products inhibiting the enzymatic activity of said kinase (FIG. 3A). After preincubation, the kinase loses a part of its phosphorylating properties up to 90%. The chemical kinases effectors such as [cAMP; Adenosine 3′-5′-cyclic monophosphate, 8-(4-Chlorophenylthio)-sodium salt (8-CPT-cAMP); anisomycin; cGMP; calmidazolium; calphostin; cdc2 peptide; genisterin; bisindolylmaleimide I (GFX); H7 H89; KN62; KT5720, ML7; protein kinase A inhibitor (PKI); staurosporine; tumor necrosis factor-alpha (TNF-α); phosbol-12-myristate-13-acetate (TPA)] were tested for their effects on TTP-dependent phosphorylation of 43K rapsyn. These molecules did not drastically alter the activity of the TTP dependent kinase, which is consequently a new type.

Effect of Zn

43K rapsyn contains two adjacent zinc finger motifs (42), and Zn²⁺ inhibits its TTP-dependent phosphorylation in a Mg²⁺ independent manner [˜70% inhibition/0.5-3 mM Zn²⁺/8 μM ³²P-TTP (FIG. 1, lane 2)].

Nature of the Amino Acids Phosphorylated With TTP

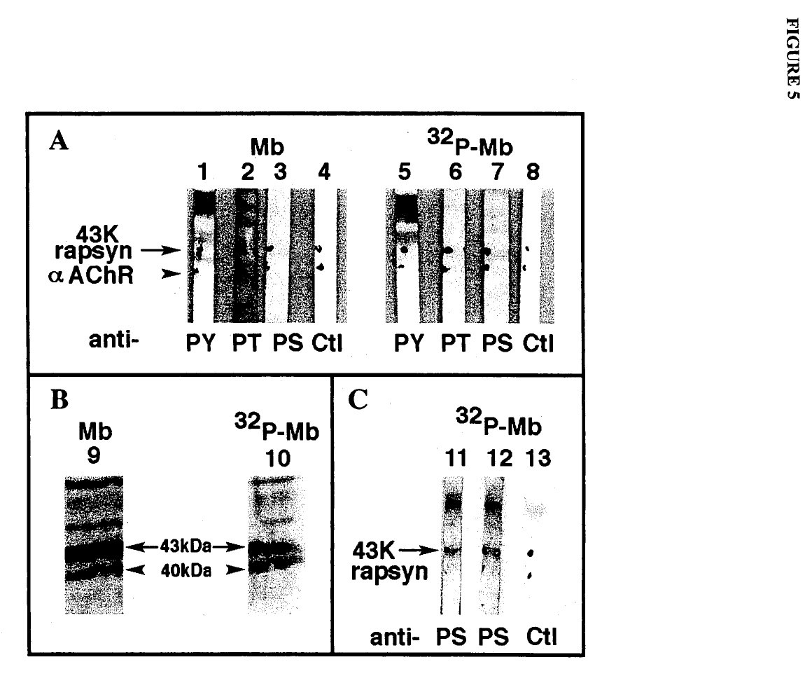

A 2D-high voltage electrophoresis of acid hydrolysates of ³²P-ATP-dependent phosphorylated 43K rapsyn (ATP-³²P-43K rapsyn) has shown that phosphorylation by PKA occurs predominantly on serine(s) (16). Similar analysis on TTP-dependent 32P-phosphorylated 43K rapsyn (TTP-³²P-43K rapsyn) showed different results with a faint radioactive signal at serine and a strong one at inorganic phosphate (Pi) (data not shown). The presence of phosphoserine has been confirmed with anti-phosphoamino acid antibodies specific to phosphoserine (PSer), phosphothreonine (PThr) and phosphotyrosine (PTyr) (FIG. 5). Equivalent amounts of control and TTP-³²P-phosphorylated membranes resolved by SDS-PAGE, were electroblotted, stained with Ponceau red (FIG. 5B, lanes 9,10) and probed with specific anti-phosphoamino acid antibodies. Anti-PTyr (FIG. 5A, lanes 1,5) strongly stained several non radioactive bands but not 43K rapsyn. This recalls the presence of in situ Tyr-phosphorylated proteins and of nAChR-associated protein tyrosine kinases (43) and suggests that Tyr is not phosphorylated in 43K rapsyn [but see (44)]. No staining of ³²P-43K rapsyn was observed with anti-PThr (FIG. 5A, lanes 2,6). Anti-PSer faintly stained 43K rapsyn both in control (FIG. 5A, lane 3) [this is consistent with the presence of in situ P-Ser in 43K rapsyn (16)], and in TTP-³²P-membrane (FIG. 5A, lane 7; FIG. 5C, lanes 11,12). A stronger staining of ³²P-43K rapsyn (lane 7 versus 3) suggests some phosphorylation on serine driven by TTP and is consistent with the reciprocal inhibitions of ATP- and TTP-dependent phosphorylations of 43K rapsyn by TTP and ATP respectively.

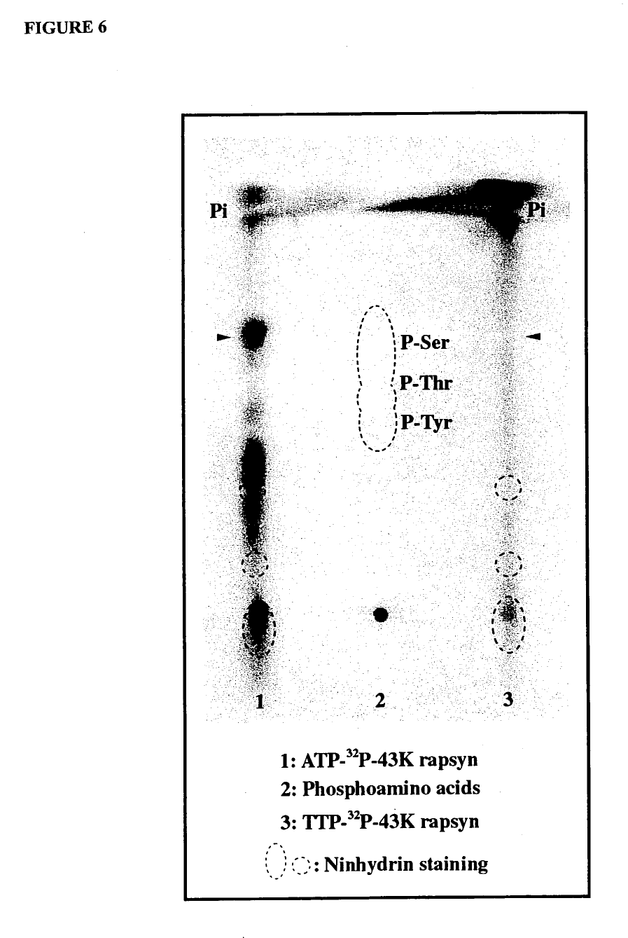

To gain insights into the unexpected high ³²Pi content in TTP-³²P-43K rapsyn hydrolysate, ATP- and TTP-³²P-43K rapsyn were simultaneously hydrolyzed with HCl and analyzed by 1D-electrophoresis. Similar ninhydrin-stained phosphopeptide patterns but different autoradiograms were obtained (FIG. 6). ATP-³²P-43K rapsyn hydrolysate (lane 1) led to high radioactivity at P-Ser (arrow head) and low radioactivity at Pi. TTP-³²P-43K rapsyn hydrolysate (lane 3) showed very faint radioactivity at P-Ser (arrow head) and high radioactivity at Pi. This confirms serine phosphorylation with ATP and suggests that phosphorylation with TTP occurs predominantly on residues other than serine and furthermore TTP driven phospholinkages are mainly acid labile.

A pH stability analysis was further performed on ATP- and TTP-³²P-43K rapsyn. SDS-PAGE gels containing both phosphoproteins were treated with TCA at 90° C., and ³²P-quantified (table I). TTP-dependent phosphorylated 43K rapsyn is acid sensitive and the ³²P-phosphate loss is a function of time in TCA (50±4 and 16±1% ³²P after 5 and 10 min versus 100±13% for control). In contrast, ATP-³²P-43K rapsyn is less sensitive (79±5 and 49±9% ³²P after 5 and 10 min versus 100±8% for control). A similar test conducted at alkaline pH (table I) showed a remarkable stability of the TTP-dependent phospholinkages (72±4% ³²P after 2 hours in 1N KOH at 46° C. versus 100±5% for control) in contrast with the ATP-driven phospholinkages (18±2 versus 100±6% ³²P in control). Thus the phosphate links elicited by ATP are acid stable and alkaline labile, a signature of O-linked phosphoamino acids phosphoserine and phosphothreonine (45). Serine is indeed phosphorylated with ATP (14; and FIG. 6). Conversely the phosphoryl linkages introduced by TTP are acid labile and alkaline stable, a characteristic of N-phosphate linkages at phosphohistidine or phospholysine (45).

TTP Causes Phosphorylation Predominantly on Histidine Residues.

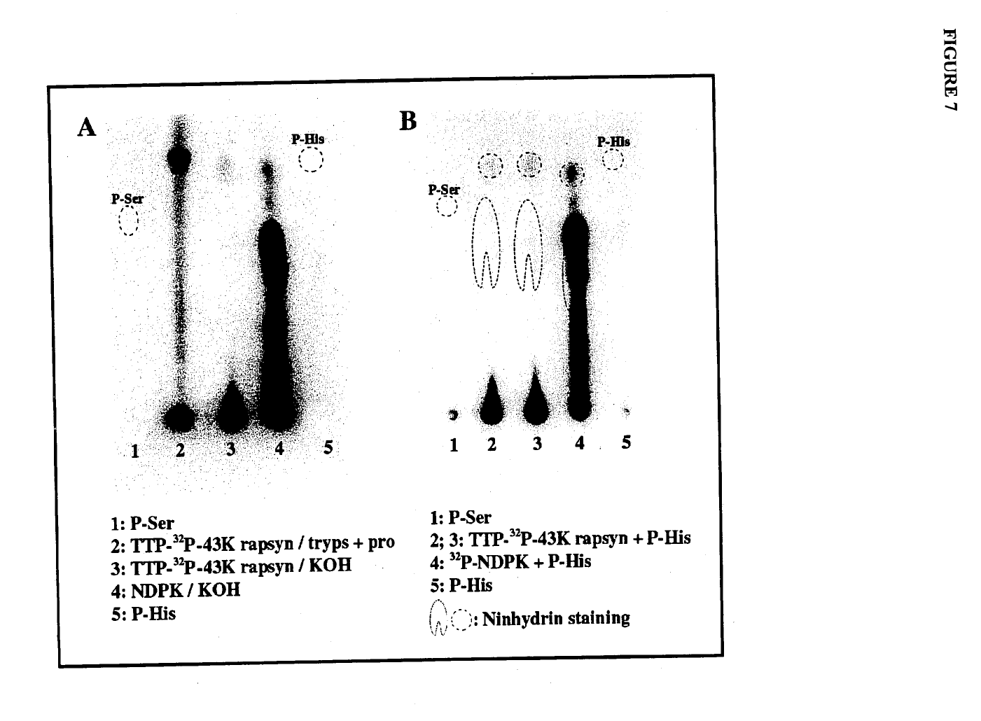

To identify the N-phosphoamino acids in TTP-phosphorylated 43K rapsyn, a TLC of 43K rapsyn hydrolysates was performed in solvent A. Nucleoside diphosphate kinase (NDPK) (46) which autophosphorylates histidine (47) was used as control (FIGS. 7A,B, lane 4). All hydrolysates (FIGS. 7A, B, lanes 2-4) displayed radioactive material migrating similarly to phosphohistidine, the highest intensity being observed with the enzymatic hydrolysate from TTP-³²P-43K rapsyn (FIG. 7A, lane 2). The low radioactivity at <<P-His>> in both 43K rapsyn (FIG. 7A, lane 3; FIG. 7B, lanes 2,3) and NDPK alkaline hydrolysates (FIGS. 7A, B, lane 4), probably derived from P-His partial destruction during hydrolysis. Added phosphohistidine (internal standard) comigrated with the radioactive spots (FIG. 7B, lanes 2-4). These results favor phosphorylations on histidine with TTP.

To assess the importance of histidine(s), nAChR-membranes were pretreated with DEPC then incubated with ³²P-TTP [DEPC modifies histidines thus preventing their subsequent phosphorylation (28)]. 43K rapsyn phosphorylation was effectively decreased in DEPC-membranes (20±2% versus 100±19% ³²P in mock membranes).

Partial tryptic digestions conducted on ATP- and TTP-³²P-43K rapsyn followed by 15% acrylamide SDS-PAGE showed one major radioactive band at ˜6.5-15 kDa for ATP- and several radioactive bands from ˜6.5 to 35 kDa for TTP-phosphorylated 43K rapsyn (data not shown). This again indicates different phosphorylation sites depending on the nature of the phosphodonor. ATP probably leads to phosphorylation mainly on one serine residue while with TTP one or several histidine residues might be mainly phosphorylated.

TTP is Not a Phosphodonor for NDPK

NDPK is a highly conserved enzyme which plays a key role in growth and metastasis control (47). As the enzyme autophosphorylates histidine and presents a broad specificity, phosphorylation was assayed with TTP. NDPK was strongly phosphorylated with ³²P-ATP but not with ³²P-TTP (data not shown).

TTP, A Phosphate Donor in the Central Nervous System (CNS)

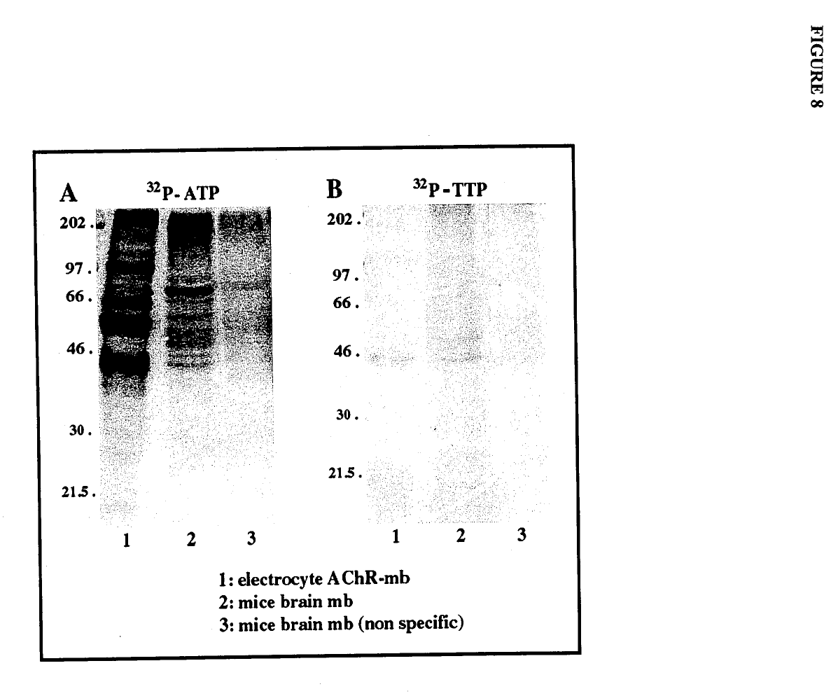

Mouse and rat brain membranes incubated with ³²P-TTP or ³²P-ATP were phosphorylated. However, as with Torpedo postsynaptic membranes (FIGS. 8A, B, lanes 1), ATP phosphorylated many proteins in mouse brain membranes (FIG. 8A, lane 2) while TTP phosphorylated very few. Two major ³²P-labelled bands were observed at ˜43-46 kDa (FIG. 8B, lane 2). Phosphorylations were partially inhibited by ATP (FIG. 8A, lane 3) or TTP (FIG. 8B, lane 3). Thus, in vitro, TTP is also a phosphodonor for proteins in the CNS.

Discussion

Endogenous PKA associated with nAChR-rich membrane preparations phosphorylate 43K rapsyn and other proteins (16). The essential role of 43K rapsyn in nAChR clustering and postsynaptic structure formation prompted us to search for a specific phosphorylation of 43K rapsyn. By immunoprecipitation and western blot analysis we have identified such phosphorylation using TTP as phosphodonor.

The TTP-Dependent Kinase(s), (a) Novel Kinase(s)

Specific phosphorylation of 43K rapsyn with TTP as phosphodonor occurs at 4-30° C., temperatures compatible with that of the sea water surrounding the Torpedo. 43K rapsyn is localized at the postsynaptic membrane inner face (5, 6) hence topologically accessible to the high cytosolic TTP content (˜4-30 nmol/g wet tissue; 24, 26). Thus, conditions necessary for a successful endogenous phosphorylation of 43K rapsyn are met supporting the notion that phosphorylation of 43K rapsyn with TTP as phosphoryl donor occurs in vivo in Torpedo electrocytes.

The phosphorylation is Mg²⁺- and TTP-dependent with characteristics of an enzymatic reaction driven by endogenous kinase(s) present in the nAChR-rich postsynaptic membrane and specific for TTP although with some affinity for ATP. They were named <<TTP-dependent-43K rapsyn kinase(s) or TTP-kinase(s)>>.

These TTP-kinase(s) seem of a novel type, different from PKA, PKC or common kinases. Their affinity is not drastically affected by inhibitors of PKA, activators (TPA) or inhibitors (calphostin, GFX) of PKC or effectors of other common kinases. The question of their classification in a new eukaryotic protein kinase family or as members of known kinase families will be solved with their identification, purification, characterization and sequencing.

Histidine Phosphorylation of 43K Rapsyn

Phosphoamino acid and antibody analysis suggest that besides some minor phosphorylation on serine residues, histidines are predominantly phosphorylated. The inhibition of TTP-dependent 43K phosphorylation by both ATP and TTP suggests that TTP-kinase might share some common phosphorylation sites with PKA. However since most of the detectable phosphoryl groups introduced by TTP are on histidine(s), and those by ATP on serine (16), the inhibition through shared serine site(s) should only account partially. The strong inhibition of phosphorylation by high concentrations of heterologous triphosphate ATP may possibly reside on the ability of TTP-kinase to recognize and link either ATP or TTP although with different affinities. In addition, a modification of the histidine(s) microenvironment brought about by phosphorylation of serine(s) might occur and result in a decrease of histidine phosphorylation by TTP-kinase(s) (Ser-406, a strong PKA consensus site, is close to His-384 and His-387).

Zn²⁺ modulates the activity of many proteins and may play a role in synaptic transmission (48) and we have shown that TTP-kinase activity is prevented by 500 μM Zn²⁺. At its C-terminus, ahead of Ser-406, 43K rapsyn displays two zinc finger motifs which could possibly be important for nAChR clustering (42, 49, 50). In addition, two conserved histidines, His-384 and His-387, are present in the zinc finger motifs. In vitro, 43K rapsyn binds Zn²⁺ probably through the two histidines (42) which consequently might become less available for an eventual phosphorylation. Binding of Zn²⁺ might also elicit conformational changes inducing a decrease of 43K rapsyn accessibility for histidine phosphorylation by TTP-kinase. If Zn²⁺ binds to 43K rapsyn in vivo, the zinc finger domain might play a role in the regulation of the protein phosphorylation state. An intrinsic sensitivity of TTP-kinase to Zn²⁺ should account only partially at these Zn²⁺ concentrations.

Tryptic digestions suggest that ATP probably leads to the phosphorylation of one main serine (possibly Ser 406, a strongly conserved PKA consensus site) while TTP may drive phosphorylation on one or several histidines. 43K rapsyn possesses thirteen histidines which are potential candidates. Ten of these residues are conserved among chick (51), human (52), mouse (53), Torpedo (54), Xenopus (55). Some of the conserved histidines have also their neighboring sequence conserved, e.g. His-154; His-239; His-256; His-384 and His-387 of the tandem zinc fingers. Highly homologous, although not totally conserved neighboring sequences of His-53; His-329; His-348; and His-353, are located in regions possibly important for 43K rapsyn functions. His-53 is present in a domain involved in 43K rapsyn self-association (56). Mutations of His-384 and His-387 reduce 43K rapsyn ability to form clusters (42). His-348 and His-353 are located between these two important regions of 43K rapsyn. The neighboring sequence RYAH of His-154 is conserved in K. aetogenes (57), N. meningitidis (58), and E. coli (59) and has been identified as a phosphorylation site essential for polyphosphate kinase activity in prokaryotes (59). The phosphate in phosphohistidine is of a high energy state and is often further transferred to an acceptor residue (on the same or another molecule), an important step in the two-component signaling mechanisms in cell regulation (60, 61). It will be of interest to identify the histidine(s) phosphorylated by TTP and determine by mutational analysis if a similar role of histidine phosphorylations can be related to 43K rapsyn phosphorylating and clustering functions in the postsynaptic domain.

TTP-Dependent Phosphorylation of 43K Rapsyn, TTP-Kinase(s) and nAChR Clustering.

43K rapsyn is present as cytosolic and membrane-attached pools in a ratio depending on tissue maturation (37). The question of a relationship between 43K rapsyn phosphorylation and its cellular compartmentations is raised.

nAChR phosphorylation has been reported in several instances (62-65). 43K rapsyn regulates tyrosine phosphorylation of several postsynaptic membrane proteins including the nAChR β-subunit (44). nAChR tyrosine phosphorylation regulates the rapid rate of receptor desensitization and may play a role in nerve-induced nAChR clustering (65-67). Two protein tyrosine kinases associated with the nAChR have been cloned in Torpedo electrocyte (43). The TTP-kinase(s) which drive specific phosphorylation(s) of 43K rapsyn predominantly on histidine(s) are also present in nAChR-rich postsynaptic membrane. Their purification (see above) will also allow further analysis of their possible involvement in the cascade responsible for nAChR phosphorylation and clustering.

Out of eighteen common protein kinase effectors tested only staurosporine, a potent but non specific protein kinase inhibitor (68), causes some inhibition. Staurosporine also inhibits agrin-induced nAChR phosphorylation and aggregation (69). This raises the question of an eventual connection between these events and 43K rapsyn phosphorylation via TTP. Agrin plays an important role in NMJ differentiation (70-72). Cotransfected 43K rapsyn causes clustering of dystroglycan, the agrin-binding component of the dystrophin glycoprotein complex (73). It also induces clustering and activation of MuSK, a synapse-associated muscle specific kinase (74-75) and component of the agrin-MuSK-MASC signaling complex responsible for nAChR clustering and postsynaptic differentiation. It will be interesting to study the influence of TTP-dependent phosphorylation on the involvement of 43K rapsyn in the agrin-dystroglycan-MuSK-MASC cascade.

Phosphorylation of 43K rapsyn through TTP also suggests the possibility of an interplay between 43K rapsyn and the thiamine pathway in excitable cells. Increased nervous activity leads to dephosphorylation from TTP and TDP to TMP and thiamine (18, 76) and deafferentation of the cerebellum decreases turnover of thiamine phosphate derivatives (77).

Extension of TTP-Dependent Phosphorylations to Other Eukaryotic Systems. TTP, a Phosphodonor for Mammalian Synaptic Proteins

Occurrence of the TTP-dependent phosphorylation of 43K rapsyn at the vertebrate NMJ remains to be defined as well as its potential role in protein-protein interactions, nAChR aggregation and stabilization at the NMJ.

Although TTP is not a phosphodonor for NDPK histidine [despite NDPK's broad specificity (47)], TTP can cause phosphorylation of proteins present in rodent central nervous membranes. TTP thus represents a valuable tool for defining a possibly novel phosphorylation pathway specific for synaptic proteins.

43K rapsyn causes clustering of co-transfected GABA_{A} receptors (78) and is present in chick ciliary ganglion neurons (51). Analysis of a possible involvement of TTP as a phosphodonor in the phosphorylation of brain receptors, chick ciliary ganglion 43K rapsyn and putative brain 43K rapsyn homologs should permit a better understanding of the molecular processes underlying synaptic functions.

The novel and specific TTP-dependent phosphorylation of 43K rapsyn highlights the possible importance of TTP-dependent phosphorylation in the modulation of synaptic organization. It also opens up a new phosphorylation pathway for synaptic proteins which differs from the more classical purine triphosphate pathway.

TTP is a donor of phosphate for endokinases in many physiological systems besides the CNS and muscle.

TTP can be a phosphodonor for the central nervous system (CNS) via endogenous kinases. Proteins present in many different CNS tissues (whole CNS, Superior cervical ganglia (SCG), neurospheres) are phosphorylated with TTP as the phosphodonoer via endogenous kinases (Nghiêm, FIG. 9).

TTP can also be a phosphodonor for proteins present in many other important physiological systems (endogenous kinase) for instance the human red blood cells (Tm, Ts), mouse immune system (bone marrow granulocytes, FIG. 11), allergenic plants (Dactylis glomerata pollen), parasites (Pm, Ps; Plasmodium falciparum) (Nghiêm, FIGS. 9 and 10). In SDS-PAGE gels, the TTP-dependent phosphorylated proteins migrate in regions similar to proteins important for the systems.

All phosphorylations were performed with 32P-TTP without any addition of exokinases. This demonstrates the presence of TTP-dependent endokinases in the tissues tested.

According to these results, the use of TTP as a donor of phosphate can be generalized to many other physiological systems besides the CNS and muscle.

FIG. 9 shows an autoradiogram obtained after TTP-dependent phosphorylation of membranes from different tissues. The regions under 30 kDa have not been examined. Molecular markers were at far left lane.

Phosphorylation of proteins from human red blood cell (HRB) membrane (Tm) occurs mainly at bands around 30-40 kDa, 70 kDa and 200 kDa). The HRB lysate (Ts) shows at least three phosphorylated bands, one around 66 kDa, and two highly phosphorylated bands in the 70 and 200 kDa regions). Comparison between phosphorylations in fractions Pm (parasite+red blood cell membrane) and Tm (human blood cell membrane) shows that the phosphorylated protein bands which are detected only in the Pm fraction (lane Pm, see for instance bands around 50, 55-60, 100, between 100 and 201 kDa, FIGS. 9a and 9b) should derive from the P. falciparum parasite. The phosphorylation patterns in lysates Ps and Ts are two weak to allow a clear cut answer. (In FIG. 9b the amount of proteins in Pm and Tm has been doubled compared to the same fractions in FIG. 9a).

Adult mouse brain membrane (A) shows phosphorylated proteins mostly at the 46-50 and 100 kDa regions. Phosphorylations can also be observed with 15 day-embryonic mouse brain membranes (E15, FIGS. 9a and 9b). The two phosphorylation patterns between Ad and E15 are not identical and might be due to age differences of the brain fractions.

Mouse stem cell neurospheres (Sa and Sf) showed phosphorylation at the 50-60 kDa regions (FIG. 9a).

Mouse superior cervical ganglion membranes© SCG) showed phosphorylation between the 40-66 kDa regions and supernates (S SCG) were also phosphorylated with TTP (phosphorylated bands between the 30 to 97 kDa).

In FIG. 9b Dactyle pollen membrane proteins are phosphorylated mainly at the 30 and 55-60 kDa regions © pollen). With the pollen lysate fraction (5 pollen), a main phosphorylated band was observed at the 55-60 kDa region (see also FIG. 10).

Control phosphorylations were performed with electrocyte membranes (co mb).

FIG. 10 shows an autoradiogram obtained after phosphorylation with 32P-TTP of Dactyle pollen proteins. Proteins in the 30 to 66 kDa regions (*) are phosphorylated at 15° C. and at 30° C. with 32P-TTP by endogenous kinases both in the water-extract (lanes 2 to 4; 9 to 11) and the pellet fractions (lanes 5 to 7; 12 to 14). Specificity of the TTP-dependent phosphorylation of the water-extract (lane E1) and of the pellet (lane PI) fractions are demonstrated by a decrease of the phosphorylation upon preincubation with cold TTP.

FIG. 11 shows TTP-dependent phosphorylation of mouse bone marrow granulocytes. In FIG. 11a, right lane (SDS-PAGE 10% acrylamide), most of the phosphorylation of the pellet fraction (C2K, obtained by centrifugation at 200×g of homogenates of granulocyte cytoplasts) resided in bands at very high molecular weight (*). Phosphorylated bands were also detected around the 66- and 97 kDa region (*). A high degree of phosphorylation was also detected at the 14.5-30 kDa region (*). This band has been extracted and further characterized by autoradiography in 12% (FIG. 11b and FIG. 11c) and 20% acrylamide (FIG. 11d) SDS-PAGE gels. FIGS. 11b and 11c showed major phosphorylation bands around 25 kDa (*). FIG. 11d showed phosphorylation at the 25 kDa region (*) but also at the 30-46 kDa region (*).

As stated above, all the detected phosphorylation in the tissues are due to endokinases which are TTP-dependent. TTP has been demonstrated to be a phosphodonor for the 43K rapsyu protein of the electrocyte, a model of neuromuscular junction. The results presented here demonstrate that TTP is also a phosphodonor for various physiological tissues important for the animals.

In previous reports we have shown that TTP is also a phosphodonor for the CNS. We extend our observations in specified tissues such as SCG. Besides the phosphorylation pattern seems dependent on age and might then be important in the differentiation process.

Bone narrow cells are also very interesting due to their potentiality in regeneration of cell lines. It is interesting that such cells are phosphorylated by TTP. A hyper-phosphorylation or a deficit in their phosphorylation might prove to be relevant to their regenerative properties.

Neurospheres are also important for their regenerative multipotentiality. They are also phosphorylated although with a weak signal in our gels (this might be due to the minute amounts of neurospheres used in the experiments).

P. falciparum is the most virulent parasite causing human malaria. P. falciparum-infected erythrocytes develop electron dense protrusions called knobs on their plasma membrane. Knobs are necessary although not sufficient for infected erythrocytes to bind to endothelial cells. The knobby phenotype may contribute to cerebral malaria (Pologe et al., 1987). A 80-90 kDa knob-associated histidine-rich protein (KAHRP) of P. falciparum which shares similarity with that present in P. Lophurae (Ravetch et al., 1984) has been correlated with the presence of knobs and sequestration (Leech et al., 1984, Pologe et al., 1987). This KAHRP shows very similar characteristics to 43K rapsyn. 43K rapsyn is located at the cyctoplasmic face of the postsynaptic membranes in electrocytes and at postsynaptic densities of the neuromuscular junctions (Nghiêm et al., 2000). The KAHRP protein is localized at the cytoplasmic face of these knobs (Pologe et al., 1987) and may play a role in cytoadherence induction (Udeinya et al., 1983). As common in malaria parasites, KAHRP contains a polyhistidine repeat structure and tandemly repeating aminoacids with a consensus motif (GlyHisHisProHis for KARH, Koide et al., 1986). Dr. Mercereau-Pujalon's unit as Institute Pasteur is involved in the study of the parasite antigens and the host-parasite interactions with the goal to develop vaccines especially with a R23 antigen. This conserved antigen contains 11 repeats with a 6 AsnHisLysSerAspSer/His/Asn,aminoacid concensus motif with His as one of the aminoacids of the motif. This antigen is recognized by opsonizing antibodies directed agains P/falciparum-infected red blood cells and recombinant R23 can induce a good protection in Saimiri sciureus monkeys (Perraut et al., 1995, 1997, in press). Phosphorylation of parasite proteins might be important in modulating their infectious or their vaccinal properties.

The essential properties of the red blood cells may be related to their degree of phosphorylation by TTP, and if red blood cells diseases or infectability can be modulated by a hyper or a deficit of the phosphorylation of their proteins.

The allergenic Dactylis glomerate pollen is abundant and widespread all over the temperate parts of the world. Two major allergens Dac g3 and Dac g4 are present in Dactylis glomerata pollen. Dac g3 (30 kDa) is cloned, sequenced and recognized by sera from many human allergic patterns (Guerin-Marchand et al.). Dac g4 (60 kDa) which is a major basic pollen allergen present in many pollen species has been purified, characterized and monoclonal antibodies to Dac g4 have been produced (Leduc-Brodard et al.). A relevant question is the relationship between the allergenicity and TTP-dependent phosphorylation of the pollen proteins.

Materials & Methods Used.

Inhibitors of proteases: (aprotimin, pefabloc, leupeptin, antipain, pepstatin A).

Postsynaptic membranes. nAChR-rich postsynaptic membranes (nAChR-membranes) were prepared from electric organs excised from freshly killed Torpedo marmorata (T.m.) (Biologie Marine, Arcachon) (Sobel et al., 1977, Hill et al., 1991).

Rodent brain, SCG, and neural spheres membrane preparation. Brain membrane preparations were performed at 4° C. Mouse and rat were anesthetized then killed by decapitation. The brains were dissected and homogenized with a teflon glass homogenizer in 5 volumes ice-cold Tris-buffer pH 7.5 containing 10% sucrose (w/w), 1 mM EDTA, 1 mM DTT and inhibitors of proteases (aprotinin, pefabloc, leupeptin, antipain, pepstatin A, PMSF). The homogenates were centrifuged at 1000 g for 5 minutes at 4° C. Thee supernatants were further centrifuged at 30,000×g for 1 hour at 4° C. The resulting pellets corresponding to the crude brain membrane fractions were homogenized in the ice-cold homogenization buffer devoided of DTT and stored at −80° C.

Mouse bone marrow granulocyte membrane preparation (4° C.). Bone marrow granulocyte cells were isolated from mouse and cytoplasts devoided of nuclei were prepared according to Wigler and Weinstein 1975 and tested for the presence of the LY-6G a marker for granulocyte and the absence of B220 a marker for lymphoid cells. Cytoplast membranes were prepared according to Wright et al., 1997 by homogenization in glass potter with 10 mM Hepes pH 7.5 buffer in the presence of 10 mM EGTA and inhibitors of proteases and centrifugation at 2000×5 min to give the pellet C2K and a supernatant which is further centrifuged at 57000×1 h to give the pellets C57K. Pellets were stored at −80° C.

Dactyle pollen fractoins: 50 mg of Dactyle pollen was rotated in 300 μl cold H₂O in the presence of inhibitors of proteases (aprotinin, pefabloc, leupeptin, antipain, pepstatin A) for 1 h and centrifuged at 14K at 4° C. for 15 min to give a supernate (extract) and a pellet fraction. The pellet fraction is resuspended in H₂O, in the presence of inhibitors of proteases (aprotinin, pefabloc, leupeptin, antipain, pepstatin A). Both fractions are stored at −80° C.

Human Red blood cells (HRB). Red blood cells are from human blood (A+) collected on citrate to prevent coagulation. The blood was kept at 4° C. for 2 to 4 weeks in 50 ml tubes then washed in RPMI 1640 (Gibco) and depleted of plasma and leukocytes. The red blood cells were centrifuged at 900×g for 10 minutes, RT, and diluted twice with RPMI. The red blood cells were then cultured in a humidified oven at 37° C. in the presence of CO₂, RPMI,+10% human serum+glucose (2 g/l), hypoxanthine (20 mg/l)+gentamycine (2.5 mg/I) and buffered With Hepes (9 g/l) and NaHCO₃ (2 g/l), pH 7.2. The red blood cell cultures were lysed in the presence of H₂O and inhibitors of proteases (aprotinin, pefabloc, leupeptin, antipain, pepsatin A) for 30 min at 4° C., centrifuged at 14K 30 min at 4° C. to give the supernatant or lysate (Ps) and pellet (Pm) fractions. Pellets were washed twice in H₂O+inhibitors of proteases. Both fractions (lysate and membrane) were stored at −80° C.

P. falciparum parasite cultures. The parasites were grown on human red blood cells. Red blood cells are from human blood (A+) collected on citrate to prevent coagulation. The blood was kept at 4° C. for 2 to 4 weeks in 50 ml tubes then washed in RPMI 1640 (Gibco) and depleted of plasma and leukocytes. The red blood cells were centrifuged at 900×g for 10 minutes, RT, and diluted twice with RPMI. The red blood cells were then cultured in a humidified oven at 37° C. in the presence of CO₂, RPMI,+10% human serum+glucose (2 g/l), hypoxanthine (20 mg/l)+gentamycine (2.5 mg/l) and buffered with Jepes (9 g/l) and HaHCO₃ (2 g/l), pH 7.2. The cultures were regularly diluted with medium containing human red blood cells to maintain a high degree of parasite growth. The cultures with high content of parasites were centrifuged at 600×g for 10 min at RT. The cell pellet was resuspended in a mixture of plasma gel and RPMI and homogenized and incubated at 37° C. for 30 min. then centrifuged at low speed (150×g, 10 min). The pellet is composed of red blood cells infected by mamre parasites and is lysed in the presence of H₂O and inhibitors of proteases (aprotinin, pefabloc, leupeptin, antipain, pepstatin A) for 30 min. at 4° C., centrifuged at 14K 30 min. at 4° C. to give the supernatant or lysate (Ps) and pellet (Pm) fractions. Pellets were washed twice in H₂O+inhibitors of proteases. Both fractions (lysate and membrane) were stored at −80° C.

Preparation of neurospheres. Neurospheres are prepared according to Reynolds and Weiss 1992; 1996 with slight modifications. Embryonic striatal cells were isolated from pregnant mice and cultured in DMEM F12 in the presence of B27 nutrient (Gibco) and EGF. Medium was change partly twice a week. Cells which float in the medium were separated from adherent cells, dissociated and maintained in culture medium with weekly passage until use.

Phosphate donors, phosphorylation and quantification. [g-³²P]-ATP (³²P-ATP) was from ICN. [g-³²P]-TTP (³²P-TTP) was synthesized (Grandfils et al., 1988). nAChR-membranes were phosphorylated with (7-8000 Ci/mol) ³²P-TTP or ³²P-ATP in 50 mM Tris-HCl pH 7.5, 5-15 mM MgCl₂, 0.08% CHAPS, inhibitors of proteases at 4°-20° C. for 60-90 minutes. Phosphorylation was stopped with SDS-sample buffer. ³²P-phosphorylated membranes were subjected to SDS-PAGE designed to separate actin, 43K rapsyn and a-nAChR, and autoradiographed (Kodak Biomax) and/or ³²P-quantified (Molecular Dynamics phosphorimager). Coomassie blue staining was performed when necessary.

Histidine Phosphorylation in Eukaryotes

In eucaryotes, phosphorylation has been estimated to occur predominantly on serine residues (˜90%), 9.9% on threonine residues and only ˜0.1% on tyrosine residues despite its key role in cell modulation (32). Phosphorylation on histidine (6%) has been mostly documented in procaryotes and often related to regulation processes (61). Fewer cases are reported in eucaryotes (79). The present invention, thus provides a surprising new means of effecting histidine phosphorylation on a synaptic protein in eukaryotic cells thus broadening the importance of histidine in eucaryotic phosphorylation.

Screening Allergenic Molecules in vitro

In another aspect of the present invention, allergenic proteins may be screened in vitro and the modulation of allergenic properties may be evaluated after phosphorylation.

In particular, and as noted above, in accordance with the present invention, TTP is used to effect phosphorylation of proteins, particularly those bearing histidine residues, for example. Thus, the present invention may be used to phosphorylate allergenic proteins of interest. For example, the allergenic proteins may be any allergenic protein that is associated with a disease or condition related to an allergy in humans. Notably, allergies caused by various pollens, particularly Dactylis glomerata may be mentioned.

Thus, this aspect of the present invention provides a method of screening in vitro an allergenic molecule of interest, which entails, a) contacting allergenic molecules of interest with TTP or a composition containing TTP under conditions effective to cause phosphorylation of the molecules by TTP; b) purifying the phosphorylated allergenic molecules; and c) evaluating a modulation of allergenic properties of the purified phosphorylated allergenic molecules.

This method permits one to evaluate and categorize allergenic molecules of interest as a function of the modulation of their allergenicity after phosphorylation. The conditions required to phosphorylate any allergenic molecule of interest are readily apparent to one skilled in the art in view of both background knowledge and the teachings described above in accordance with the present invention. Also, the purification of the phosphorylated allergenic molecule may be effected by one skilled in the art in view of both background knowledge in combination with the present invention. Finally, means for detecting and evaluating allergenic properties of allergenic substances are also well known to those skilled in the art.

Compositions Containing TTP

As described above, in accordance with the present invention, any composition containing TTP is explicitly contemplated. These compositions may include solid or liquid compositions and may be in a form suitable for any form of administration, including, but not limited to oral, intravenous injection, intramuscular injection and suppository.

The compositions may be a combination of TTP and a carrier, such as dextrose 5% saline for injection, or magnesium stearate for oral administration, for example. However, other biochemically active ingredients may be present, such as other phosphorylating agents, vitamins, cofactors, minerals, trace metals, and natural products or extracts of natural products, such as ginseng, echinacea, or gingko biloba. In particular, the present invention explicitly includes vitamin and/or antioxidant compositions also containing TTP. Such compositions may either include TTP in lieu of thiamine (vitamin B1) or thiamine pyrophosphate or in combination with either or both of these constituents.

The amount of TTP used in these compositions may be a described hereinabove on a ug or mg/kg of body weight basis or may be from one tenth to 1,000 times the recommended daily dosage of thiamine, for example, with a view toward optimizing a desired concentration range of TTP in any particular area of interest in the body, if necessary, in order to ensure adequate phosphorylation of cells. Such an optimization of concentration is within the ambit of one skilled in the art in view of the present specification.

Conclusion

The present inventors have now demonstrated for the first time the phosphorylation of proteins by thiamine triphosphate (TTP), a triphosphate component distinct from ATP or GTP. The protein kinase(s) appears to be of a novel type. The amino acid phosphorylated is also not uncommon in eucaryotes since it is mainly histidine. It has also now been demonstrated that the protein target in this phosphorylation is 43K rapsyn which is specifically present in postsynaptic membranes and essential for the synapse to function properly. This new type of TTP-dependent phosphorylation is not restricted to 43K rapsyn but is also observed with mouse and rat brain membranes. This affords broad and a more general use of TTP as a phosphate donor in a novel phosphorylation pathway, and also provides a means of screening or evaluating allergenic molecules of interest by phosphorylation.

Clearly, numerous modifications and variations of the present invention are possible in light of the above teachings. It is therefore to be understood that many changes and modifications may be made to the above-described embodiments without departing from the spirit and the scope of the present invention.

TABLE I
Determination of the predominant phosphate links in ATP- and TTP-dependent
phosphorylated 43K rapsyn by chemical stability and phosphoamino acid (Paa) analysis

| | 43K-rapsyn/ATP | 43K-rapsyn/TTP |
|---|---|---|
| Treatment | % ³²P | % ³²P |
| SDS-PAGE gel + acid | | |
| Ctl/Tris, 90° C. 10 min | 100 ± 8 | 100 ± 13 |
| 16% TCA, 90° C. 5 min | 79 ± 5 | 50 ± 4 |
| 16% TCA, 90° C. 10 min | 49 ± 9 | 16 ± 1 |
| P-aa stability | acid-stable | acid-stable |
| Putative Paa (ref. 45) | P-Cys, P-Ser, P-Thr, P-Tyr | P-Arg, P-His, P-Lys, P-Asp, P-Glu |
| PVDF blot + base | | |
| Ctl/water, 46° C. 120 min | 100 ± 6 | 100 ± 5 |
| 1 N KOH 46° C. 40 min | 41 ± 1 | 96 ± 3 |
| 1 N KOH 46° C. 120 min | 18 ± 2 | 72 ± 4 |
| P-aa stability | base-labile | base-stabile |
| Putative Paa (ref. 45) | P-Arg, P-Ser,, P-Thr, P-Asp, P-Glu | P-Cys, P-His, P-Lys, P-Tyr |
| Paa/pH-stability *(ref. 45) | P-Ser and/or P-Thr | P-His and/or P-Lys |
| Paa analysis (text; FIGS. 6, | P-Ser | P-His |
| 7) | | |

Values: (standard mean ± standard deviation) of % ³²P in 43K rapsyn phosphorylated with ³²P-ATP (43K-rapsyn/ATP) or ³²P-TTP (43K-rapsyn/TTP) and incubated in TCA or KOH relative to control mock treated counterparts.
*The Paa determined by pH-stability analysis are putative Paa listed in both acid- and base-stability analysis (underlined).

References

1. Nghiêm, H. O., and Changeux, J. P. (1993) The 43KDa protein (43 K). In Guidebook to the cytoskeletal, extracellular matrix and adhesion proteins. (Vale, R., and Kreis, T., eds), pp. 95-97, Sambrook & Tooze, Oxford

2. Nghiêm, H. O. (in press) The 43 K protein or 43K-rapsyn protein. In Guide book to the cytoskeletal and motor proteins (Kreis, T., and Vale, R., eds), Sambrook & Tooze, Oxford

3. Sobel, A., Weber, M., and Changeux, J. P. (1977) Large scale purification of the acetylcholine receptor protein in its membrane-bound and detergent extracted forms from Torpedo marmorata electric organ. Eur. J. Biochem. 80, 215-224

4. Neubig, R. R., Krodel, E. K., Boyd, N. D., and Cohen, J. B. (1979) Acetylcholine and local anesthetic binding to Torpedo nicotinic postsynaptic membranes after removal of nonreceptor peptides. Proc. Natl. Acad. Sci. USA. 76, 690-694

5. Nghiêm, H. O., Cartaud, J., Dubreuil, C., Kordeli, C., Buttin, G., and Changeux, J. P. (1983) Production and characterization of a monoclonal antibody directed against the 43,000 M.W. v1 polypeptide from Torpedo marmorata electric organ. Proc. Natl. Acad. Sci. USA 80, 6403-6407

6. Sealock, R., Wray, B. E.; and Froehner, S. C. (1984) Ultrastructural localization of the Mᵣ 43,000 protein and the acetylcholine receptor in Torpedo postsynaptic membranes using monoclonal antibodies. J. Cell. Biol. 98, 2239-2244

7. Froehner, S. C., Gulbrandsen, V., Hyman, C., Jeng, A. Y., Neubig, R. R., and Cohen, J. B. (1981) Immunofluorescence localization at the mammalian neuromuscular junction of the Mᵣ 43,000 protein of Torpedo postsynaptic Membranes. Proc. Natl. Acad. Sci. USA 78, 5230-5234

8. Gautam, M., Noakes, P. G., Mudd, J., Nichol, M., Chu, G. C., Sanes, J. R., and Merlie, J. P. (1995) Failure of postsynaptic specialization to develop at neuromuscular junctions of rapsyn-deficient mice. Nature 377, 232-236

9. Krikorian, J. G. and Bloch, R. J. (1992) Treatments that extract the 43K protein from acetylcholine receptor clusters modify the conformation of cytoplasmic domains of all subunits of the receptor. J. Biol. Chem. 267, 9118-9128

10. Huganir, R. L. and Greengard, P. (1990) Regulation of neurotransmitter receptor desensitization by protein phosphorylation. Neuron 5, 555-567

11. Walsh, D. A., Newsholme, P., Cawley, K. C., Van, P. S., and Angelos, K. L. (1991) Motifs of protein phosphorylation and mechanisms of reversible covalent regulation. Physiological Reviews 71 (1), 285-304

12. Cohen, P., Campbell, D. G., Dent, P., Gomez, N., Lavoinne, A., Nakielny, S., Stokoe, D., Sutherland, C., and Traverse, S. (1992) Dissection of the protein kinase cascades involved in insulin and nerve growth factor action. Biochemical Society Transactions 20 (3), 671-674

13. Roche, K. W., Tingley, W. G. and Huganir, R. L. (1994) Glutamate receptor phosphorylation and synaptic plasticity. Curr opin. Neurol. 4, 383-388

14. Karin, M., and Hunter, T. (1995) Transcriptional control by protein phosphorylation: signal transmission from the cell surface to the nucleus. Curr. Biol. 5/7, 747-757

15. Carr, C., McCourt, D., and Cohen, J. (1987) The 43-kilodalton protein of Torpedo nicotinic postsynaptic membranes: purification and determination of primary structure. Biochemistry 26, 7090-7102

16. Hill, J. A., Nghiêm, H. O., and Changeux, J. P. (1991) Serine-specific phosphorylation of nicotinic receptor-associated 43K protein. Biochemistry 30, 5579-5585

17. Glass, D. J., and Yancopoulos, G. D. (1997) Sequential roles of agrin, MuSK and rapsyn during neuromuscular junction formation. Curr. Op. in Neurobiol. 7, 379-384

18. Cooper, J. R., and Pincus, J. H. (1979) The role of thiamine in the nervous tissue. Neurochem. Res. 4, 223-239

19. Yamashita, H., Zhang, Y. X. and Nakamura, S. (1993) The effects of thiamin and its phosphate esters on dopamine release in the rat striatum. Neurosc. Lett. 158 (2), 229-231

20. Bettendorff, L. (1994) Thiamine in excitable tissues: reflexions on a non-cofactor role. Metab. Brain Dis. 9, 183-210

21. Bettendorff, L., Peeters, M., Wins, P., and Schoffeniels, E. (1993) Metabolism of thiamine triphosphate in rat brain: correlation with chloride permeability. J. Neurochem. 60, 423-434

22. Kawasaki, T. (1992) Vitamin B1: Thiamine. In Modern Chromatographic of analysis of vitamins (De Leenhert, A. P., Lambert, W. E., and Nelis, H. J., eds) pp. 319-354, Marcel Dekker, Inc, New York

23. Bettendorff, L., Peeters, M., Jouan, C., Wins, P., and Schoffeniels, E. (1991) Determination of thiamin and its phosphate esters in cultured neurons and astrocytes using an ion-pair reversed phase high performance liquid chromatographic method. Anal. Biochem. 198, 52-59

24. Eder, L., and Dunant, Y. (1980) Thiamine and cholinergic transmission in the electric organ of Torpedo. I. Cellular localization and functional changes of thiamine and thiamine phosphate esters. J. neurochem. 35, 1278-1286

25. Egi, Y., Koyama, S., Shikata, H., Yamada, K., and Kawasaki, T. (1986) Content of thiamine phosphate esters in mammalian tissues—An extremely high concentration of thiamine triphosphate in pig skeletal muscle. Biochem. Int. 12, 385-390

26. Bettendorff, L., Michel-Cahay, C., Grandfils, C., De Rycker, C., and Schoffeniels, E. (1987) Thiamine triphosphate and membrane-associated thiamine phosphatases in the electric organ of Electrophorus electricus. J. Neurochem. 49, 495-502

27. Grandfils, C., Bettendorff, L., de Rycker, C., and Schoffeniels, E. (1988) Synthesis of (gamma-³²P) thiamine triphosphate. Anal. Biochem 169(2), 274-278

28. Miles, E. W. (1977) Modifications of histidyl residues in proteins by diethylpyrocarbonate. In Methods Enzymol. (Hirs, C. H. W., and Timasheff, S. N., eds), Vol 47, part E, pp. 431-442, Academic Press, N.Y.

29. Cortay, J. C., Ricul, B., Duclos, B., and Cozzone, A. J. (1986) Characterization of the phosphoproteins of Escherichia coli cells by electrophoretic analysis. Eur. J. Biochem. 159, 227-237

30. Turner, A. M., and Mann, N. H. (1986) Protein phosphorylation in Rhodomicrobium vannielii. J. Gen. Microbiol. 132, 3433-3440

31. Kamps, M. P., and Sefton, B. M. (1989) Acid and base hydrolysis of phosphoproteins bound to immobilon facilitates analysis of phosphoamino acids in gel-fractionated proteins. Anal. Biochem. 176, 22-27

32. Cooper, J. A., Sefton, B. H., and Hunter, T. (1983) Detection and quantification of phosphotyrosine in proteins. In Methods in Enzymology (Corbin, J. D., and Hardman, J. G., eds) Vol. 99, part F, pp. 387-402, Academic Press, New York

33. Smith, R. A., Halpern, R. M., Bruegger, B. B., Dunlap, A. K., and Fricke, O. (1978) Chromosomal protein phosphorylation on basic amino acids. In Methods Cell Biol. (Stein, G., Stein, J., and Kleinsmith, L. J., eds) Vol. 19, pp. 153-159, Academic Press, N.Y.

34. Wei, Y. F., and Matthews, H. R. (1991) Identification of phosphohistidine in proteins and purification of protein-histidine kinases. In Methods in Enzymology (Hunter, T,. and Sefton, B. M., eds) Vol. 200, part A, pp. 388-414, Academic Press, San Diego

35. Towbin, H., Staehelin, T., and Gordon, J. (1979) Electrophoretic transfer of proteins from polyacrylamide gels to nitrocellulose sheets: procedure and some applications. Proc. Natl. Acad. Sci. (USA) 76 (9), 4350-4354

36. Nghiêm, H. O. (1988) Miniaturization of the immunoblot technique. rapid screening for the detection of monoclonal and polyclonal antibodies. J. Immunol. Meth. 111, 137-141

37. Nghiêm, H. O., J. Hill, and Changeux, J. P. (1991) Developmental changes in the subcellular distribution of the 43K (v1) polypeptide in Torpedo marmorata electrocyte: support for a role in acetylcholine receptor stabilization. Development 113, 1059-1067

38. Krebs, E. J., and Beavo, J. A. (1979) Phosphorylation-dephosphorylation of enzymes. In Annu. Rev. Biochem. (Snell, E. E., ed, Boyer, P. D., Meister, A., and Richardson, C. C., eds) Vol. 48, pp. 923-959, Annu. Rev. Inc, Palo Alto

39. Hunter, T., and Cooper, J. A. (1985) Protein-tyrosine kinases. In Annu. Rev. Biochem. (Richardson, C. C., Boyer, P. D., Dawid I. B., and Meister, A., eds) Vol. 54, pp. 897-930, Annu. Rev. Inc, Palo Alto

40. Bairoch, A., Bucher, P., and Hofmann, K. (1997) The prosite database, its status in 1997. Nucl. Ac. Res. 25/1: 217-221

41. Kreegipuu, A., Blom, N., and Brunak, S. (1999) PhosphoBase, a database of phosphorylation sites: release 2.0. Nucleic Acids Res. 27(1), 237-239

42. Scotland, P. B., Colledge, M., Melnikova, I., Zhengshan, D., and Froehner, S. C. (1993) Clustering of the acetylcholine receptor by the 43-kD protein: involvement of the zinc finger domain. J. Cell Biol. 123, 719-728

43. Swope, S. L. and Huganir, R. L. (1993) Molecular cloning of two abundant protein tyrosine kinases in Torpedo electric organ that associate with the acetylcholine receptor. J. Biol. Chem. 268/33, 25152-25161

44. Qu, Z., Apel, E. D., Doherty, C. A., Hoffman, P. W., Merlie, J. P., and Huganir, R. L. (1996) The synapse-associated protein rapsyn regulates tyrosine phosphorylation of proteins colocalized at nicotinic acetylcholine receptor clusters. Molecular & Cellular Neurosciences 8(2-3), 171-184

45. Duclos, B., Marcandier, S., and Cozzone, A. J. (1991) Chemical properties and separation of phosphoamino acids by thin-layer chromatography and/or electrophoresis. In Methods in Enzymology (Hunter, T. and Sefton, B. M., eds) Vol. 201, part B, pp. 10-21, Academic Press, San Diego

46. Mesnildrey, S., Agou, F., Karlsson, A., Deville Bonne, D., and Veron, M. (1998) Coupling between catalysis and oligomeric structure in nucleoside diphosphate kinase. J. Biol. Chem. 273, 4436-4442

47. Parks, R. E. J., and Agarwal, R. P. (1973) Nucleoside diphosphokinases. In The Enzymes (Boyer, E. D., ed) Vol. 8, part A, pp. 307-334, Academic Press, New York

48. Huang, E. P. (1997) Metal ions and synaptic transmission: think zinc. Proc. Natl. Acad. Sci. (USA,) 94 (25), 13386-13387

49. Bezakova, G., and Bloch, R. J. (1998) The zinc finger domain of the 43KDa receptor-associated protein, rapsyn. Role in nAChR clustering. Mol. & Cell. Neurosc. 11(5-6): 274-288

50. Wang, Z. Z., Mathias, A., Gautam, M., and Hall, Z. W.(1999) Metabolic stabilization of muscle nicotinic acetylcholine receptor by rapsyn. J. Neurosc. 19(6):1998-2007

51. Burns, A. L., Benson, D., Howard, M. J., and Marjiotta, J. F., (1997) Chick ciliary ganglion neurons contain transcripts coding for acetylcholine receptor-associated protein at synapses (rapsyn). J. Neurosc. 17/13, 5016-5026

52. Buckel, A., Beeson, D., James, M., and Vincent, A. (1996) Cloning of cdna encoding human rapsyn and mapping of the rapsn gene locus to chromosome 11p11.2-p11.1. Genomics 35, 613-616

53. Frail, D. E., McLaughlin, L. L., Mudd, J., and Merlie, J. P. (1988) Identification of the mouse muscle 43,000-dalton acetylcholine receptor-associated protein (RAPsyn) by cDNA cloning. J. Biol. Chem. 263, 15602-15607

54. Frail, D. E., Mudd, J., Shah, V., Carr, C., Cohen, J. B., and Merlie, J. P. (1987) cDNAS for the postsynaptic 43-kDa protein of Torpedo electric organ encode two proteins with different carboxyl termini. Proc. Natl. Acad. Sci. U.S.A. 84, 6302-6306

55. Baldwin, T. J., Theriot, J. A., Yoshihara, C. M., and Burden, S. J. (1988) Regulation of transcript encoding the 43K subsynaptic protein during development and after denervation. Development 104, 557-564

56. Ramarao, M. K., and Cohen, J. (1998) Mechanism of nicotinic nAChR cluster formation by rapsyn. Proc. Natl. Acad. Sci. U.S.A. 95, 4007-4012

57. Kato, J., Yamamoto, T., Yamada, K., and Ohtake, H. (1993) Cloning, sequence and characterization of the polyphosphate kinase-encoding gene (ppk) of Klebsellia aeroegenes. Gene 137, 237-242

58. Tinsley, C. R., and Gotschlich, E. C. (1995) Cloning and characterization of the meningococcal polyphosphate kinase gene: production of polyphosphate synthesis mutants. Infect. Immun. 63, 1624-1630

59. Kumble, K. D., Ahn, K., and Komberg, A. (1996) Phosphohistidyl active sites in polyphosphate kinase of E. coli. Proc. Natl. Acad. Sci. U.S.A. 93, 14391-14395

60. Stock, J. B., Stock, A. M., and Mottonen, J. M. (1990) Signal transduction in bacteria. Nature 344, 395-400

61. Alex, L. A., and Simon, M. I. (1994) Protein histidine kinases and signal transduction in prokaryotes and eukaryotes. Trends Genet. 10, 133-138

62. Huganir, R. L., and Greengard, P. (1983) cAMP-dependent protein kinase phosphorylates the nicotinic acetylcholine receptor. Proc. Natl. Acad. Sci. U.S.A. 80, 1130-1134

63. Huganir R. L., Miles, K., and Greengard, P. (1984) Phosphorylation of the nicotinic acetylcholine receptor by an endogenous tyrosine-specific protein kinase. Proc. Natl. Acad. Sci. U.S.A. 81, 6968-6972

64. Safran, A., Sagi-Eisenberg, R., Neumann, D., and Fuchs, S. (1987) Phosphorylation of the acetylcholine receptor by protein kinase C and identification of the phopshorylation site within the δ subunit. J. Biol. Chem. 262, 10506-10510

65. Hopfield, J. F., Tank, D. W., Greengard, P., and Huganir, R. L. (1988) Functional modulation of acetylcholine receptor by tyrosine phosphorylation. Nature 336, 677-680

66. Qu, Z., Moritz, E., and Huganir, R. L. (1990) Regulation of tyrosine phosphorylation of the nicotinic acetylcholine receptor at the rat neuromuscular junction. Neuron 2, 367-378

67. Wallace, B. G., Qu, Z., and Huganir, R. L. (1991) Agrin induces phosphorylation of the nicotinic acetylcholine receptor. Neuron 6, 869-878

68. Rüegg, U. T., and Burgess, G. M. (1989) Staurosporine, K-252 and UCN-01: potent but non specific inhibitors of protein kinases. Trends in Pharmac. Sc. 10, 218-220

69. Wallace, B. G. (1994) Staurosporine inhibits agrin-induced acetylcholine receptor phosphorylation and aggregation. J. Cell Biol. 125, 661-668

70. McMahan U. J. (1990). The agrin hypothesis. In: Brain. Cold Spring Harbor Symp. Quant. Biol. LV, 407-418.

71. Ferns, M. J. and Hall, Z. W. (1992) How many agrins does it take to make a synapse? Cell 70, 1-3

72. Gautam, M., Noakes, P. G., Moscoso, L., Rupp, F., Scheller, R. H., Merlie, J. P., and Sanes, J. R. (1996) Defective neuromuscular synaptogenesis in agrin-deficient mutant mice. Cell 85, 525-535

73. Apel, E. D., Roberds, S. L., Campbell, K. P., and Merlie, J. P. (1995) Rapsyn may function as a link between the acetylcholine receptor and the agrin-binding dystrophin-associated glycoprotein complex. Neuron 15, 115-126

74. Gillespie, S. K. H., Balasubramanian, S., Fung, E. T., and Huganir, R. L. (1996) Rapsyn clusters and activates the synapse-specific receptor tyrosine kinase MuSK. Neuron 16, 953-962

75. Glass, D. J., Bowen, D. C., Stitt, T. N., Radziejewski, C., Bruno, J., Ryan, T. E., Gies, D. R., Shah, S., Mattsson, K., Burden, S. J., DiStefano, P. S., Valenzuela, D. M., DeChiara, T. M. and Yancopoulos, G. D. (1996) Agrin acts via a MuSK receptor complex. Cell 85, 513-523

76. Bettendorff, L., Schoffeniels, E., Naquet, R., Silva, B. C., Riche, D., and Menini, C. (1989) Phosphorylated thiamine derivatives and cortical activity in the baboon Papio papio: effect of intermittent light stimulation. J. Neurochem. 53 (1), 80-87

77. Nauti, A., Patrini, C., Reggiani, C., Merighi, A., Bellazzi, R., and Rindi, G. (1997) In vivo study of the kinetics of thiamine and its phosphoesters in the deafferented rat cerebellum. Metab. Brain Dis. 12(2), 145-160

78. Yang, S. H., Armson, P. F., Cha, J., and Phillips, W. D., (1997) Clustering of GABA_{A} receptors by rapsyn/43 kD protein in vivo. Mol. Cell. Neurosc. 8, 430-438

79. Matthews, H. R. (1995) Protein kinases and phosphatases that act on histidine, lysine, or arginine residues in eukaryotic proteins: a possible regulator of the mitogen-activated protein kinase cascade. Pharmac. Ther. 67, 323-350

All references cited above are incorporated herein by reference in their entirety.

What is claimed is:

1. A composition, comprising thiamine triphosphate, and a carrier.

2. The composition of claim 1, which is in unit dosage form.

3. The composition of claim 1, which is in a form suitable for oral, intramuscular or intravenous administration to a mammal.

4. The composition of claim 1, comprising thiamine triphosphate in an amount effective to increase eucaryotic cellular phosphorylation level.

5. The composition of claim 4, which further comprises one or more other biochemically active ingredients.

6. The composition of claim 5, wherein said one or more other biochemically active ingredients comprises vitamins, cofactors, minerals, trace metals or natural products or extracts thereof.

7. A method of treating a mammal having a pathology associated with an under-phosphorylation of a post-synaptic protein or having a deficit in the formation of functional motor endplates, comprising administering an effective amount of thiamine triphosphate to the patient.

8. The method of claim 7, wherein the thiamine triphosphate is administered in the form of a pharmaceutically acceptable composition containing the thiamine triphosphate and a pharmaceutically acceptable carrier or diluent.

9. The method of claim 7, wherein said mammal is human.

10. A method of treating cell membranes or cytoskeleton of cells which are deficient in phosphorylated histidine residues, in vivo, comprising contacting the membranes with an amount of thiamine triphosphate effective to increase the amount of phosphorylated histidine residues in a protein contained in said cell membranes or cytoskeleton of said cells.

11. The method of claim 10, wherein said deficient phosphorylated histidine residues are rapsyn.

12. A method of phosphorylating rapsyn, comprising contacting rapsyn with thiamine triphosphate to thereby phosphorylate the rapsyn.

13. A kit for detecting specific phosphorylation of histidine residues in a protein, comprising:
(a) radioactively labeled thiamine triphosphate,
(b) non-radioactively labeled thiamine triphosphate,
(c) reagents for transfer of the thiamine triphosphate,
(d) a purified protein containing TTP dependent phosphorylatable histidine residues,
(e) a protein containing non TTP-dependent phosphorylatable histidine residues, and
(f) optionally, antiphosphoamino acid antibodies.

14. A method of quantifying the level of phosphorylation of cytoskeleton of or the membranes of eucaryotic cells, comprising:
(a) purifying the membranes or the cytoskeleton from a eukaryotic cell sample obtained from a patient,
(b) incubating the membranes or the cytoskeleton with thiamine triphosphate,
(c) comparing the incorporation of exogenous phosphate with a control, and
(d) determining the presence or absence of phosphorylated histidine residues in a protein in the sample.

15. A method of phosphorylation, comprising contacting procaryotic or eucaryotic cells with thiamine triphosphate to transfer a phosphate group from the thiamine triphosphate to a phosphate acceptor group of the cells.

16. The method of claim 15, wherein the phosphate acceptor group of the cells is a histidine residue of a cellular protein.

17. A process of purifying of a protein extract carrying a TTP dependent kinase activity, which comprises:
a) obtaining an extract from eucaryotic tissue,
b) separating cytosol from membranes, and
c) identifying one or more components of the extract exhibiting kinase activity.

18. A purified protein extract having a new TTP dependent kinase activity capable of transfering a phosphate group to histidine residues of a protein.

19. A method of effecting phosphorylation in eucaryotic cells, which comprises effecting said phosphorylation with thiamine triphosphate as a phosphate donor.

20. The method of claim 19, wherein a phosphate acceptor group in said eucaryotic cells for said phosphorylation is one or more histidine residues of a cellular protein.

21. The method of claim 19, wherein said eucaryotic cells are human cells.

22. The method of claim 19, which is in vitro.

23. The method of claim 19, which is in vivo.

24. The method of claim 23, which is effected in order to treat a disease or condition associated with under-phosphorylation of cells or cellular membranes in humans.

25. The method of claim 24, wherein said disease or condition is related to underphosphorylation of a post-synaptic protein.

26. The method of claim 24, wherein said disease or condition is related to a deficit in the formation of functional motor endplates.

27. The method of claim 24, which effects treatment of a neuronal disease or condition.

28. The method of claim 24, which effects treatment of a muscular disease or condition.

29. A method of screening an allergenic molecule of interest in vitro which comprises:
a) contacting said allergenic molecule of interest with TTP or a composition containing TTP under conditions effective to cause phosphorylation of the molecule;
b) purifying the phosphorylated allergenic molecule; and
c) detecting and evaluating a modulation of allergenic properties of the purified phospharylated allergenic molecule.

30. The method of claim 29, wherein said allergenic molecule of interest is contained in pollen.

31. The method of claim 30, wherein said pollen is Dactylis glomerata.
